(19) United States
(12) Patent Application Publication
(10) Pub. No.: US 2013/0231346 A1
(43) Pub. Date: Sep. 5, 2013

(54) METHODS OF TREATING CANCER

(71) Applicants: Tona M. Gilmer, Research Triangle Park, PA (US); Peter F. Lebowitz, Radnor, PA (US); Li Liu, Collegeville, PA (US); Jolly Mazumdar, Collegeville, PA (US)

(72) Inventors: Tona M. Gilmer, Research Triangle Park, PA (US); Peter F. Lebowitz, Radnor, PA (US); Li Liu, Collegeville, PA (US); Jolly Mazumdar, Collegeville, PA (US)

(73) Assignee: GLAXOSMITHKLINE INTELLECTUAL PROPERTY (NO.2) LIMITED, Middlesex (GB)

(21) Appl. No.: 13/988,054

(22) PCT Filed: Nov. 17, 2011
(86) PCT No.: PCT/US11/61135

§ 371 Date: May 17, 2013

Related U.S. Application Data
(60) Provisional application No. 61/414,521, filed on Nov. 17, 2010.

(51) Int. Cl. A61K 31/519; A61K 31/415; A61K 45/06; A61K 31/501; A61K 31/439

(52) U.S. Cl. CPC A61K 31/519; A61K 31/501; A61K 31/439; A61K 45/06; A61K 31/415
USPC 514/252.04; 514/264.1; 514/291

(57) ABSTRACT

Methods for treating a human with cancer comprise administering a therapeutically effective amount of at least one MEK inhibitor and at least one mTOR inhibitor to said patient, wherein said patient has at least one mutation in at least one Ras protein or gene encoding at least one Ras protein and/or wherein said patient has at least one mutation, deletion or insertion in LKB1/STK11.

### FIELD OF THE INVENTION

The present invention relates to a method of treating cancer in a mammal.

### BACKGROUND OF THE INVENTION

Effective treatment of hyperproliferative disorders, including cancer, is a continuing goal and unmet medical need in the oncology field. Generally, cancer results from the deregulation of the normal processes that control cell growth, cell division, differentiation and apoptotic cell death, among others. One such process involves kinase regulation of apoptosis and cellular signaling from growth factor receptors at the cell surface to the nucleus (Crews and Erikson, Cell, 74:215-17, 1993).

A large family of enzymes is the protein kinase enzyme family. There are about 500 different known protein kinases. Protein kinases serve to catalyze the phosphorylation of an amino acid side chain in various proteins by the transfer of the γ-phosphate of the ATP-Mg²⁺ complex to said amino acid side chain. These enzymes appear to control the majority of the signaling processes inside cells, thereby governing cell function, growth, differentiation and apoptosis through reversible phosphorylation of the hydroxyl groups of serine, threonine and tyrosine residues in proteins. Studies have shown that protein kinases regulate many cell functions, including signal transduction, transcriptional regulation, cell motility, and cell division. Several oncogenes have also been shown to encode protein kinases, suggesting that kinases play a role in oncogenesis.

For example, activation of Raf-MEK-ERK signal transduction pathway in cancer, particularly colorectal cancer, pancreatic cancer, lung cancer, breast cancer and the like, has been observed.

The ras family of oncogenes (K-ras, H-ras, and N-ras) encode for membrane proteins possessing GTPase activity. These proteins are involved in cellular signal transduction. Specific point mutations, usually within the ras codons 12, 13, or 61, can result in the activation of these protooncogenes and result in subsequent neoplasia (Bos, J. L., 1989, Can. Res. 49:4682-4689). The frequency with which ras mutations occur varies among different tumor types, although not all have been tested. Studies indicate that approximately 40-50% of colon cancers exhibit a mutation in the c-K-ras gene, with 86% of these mutations occurring at codons 12 and 13 (Bos, J. L. et al., 1987, Nature 327: (6120)293-7, Vogelstein B. et al., 1988, N. Engl. J. Med. 319:525-532). Ras mutations result in increased cell proliferation due to decreased intrinsic GTP-ase activity of the Ras protein.

The mammalian target of rapamycin (mTOR), also known as FK506 binding protein 12 rapamycin associated protein q (FRAP1), is a protein which in humans is encoded by the FRAP1 gene. mTOR is a serine/threonine protein kinase that regulates cell growth, cell proliferation, cell motility, cell survival, protein synthesis, and transcription.

Current research indicates that mTOR integrates the input from multiple upstream pathways including insulin, growth factors (such as IGF-1 and IGF-2), and mitogens. mTOR also functions as a sensor of cellular nutrient and energy levels and redox status. The disregulation of the mTOR pathway is implicated as a contributing factor to various human disease processes, especially various types of cancer. Rapamycin is a bacterial natural product that can inhibit mTOR through association with its intracellular receptor FKBP12. The FKBP12-rapamycin complex binds directly to the FKBP12-Rapamycin Binding (FRB) domain of the mTOR.

mTOR has been shown to function as the catalytic subunit of two distinct molecular complexes in cells, mTORC1 and mTORC2. mTOR inhibitors are already used in the treatment of transplant rejection. They are also beginning to be used for the treatment of cancer. mTOR inhibitors may also be useful for treating several age-associated diseases.

Germline mutation in serine/threonine kinase 11 (STK11, also called LKB1) results in Peutz-Jeghers syndrome, characterized by intestinal hamartomas and increased incidence of epithelial cancers. (Hongbin, et al. Nature (2007) 448:807-810 and Hearle, et al., Clin. Cancer Res. (2006) 12:3209-3215). Somatic LKB1 mutations have been reported in some primary human lung adenocarcinomas and has been shown to modulate cell differentiation and metastasis in Kras mutated lung cancer (Hongbin, et al.)

It would be useful to provide novel methods of treatment for an individual with cancer having at least one Ras protein mutation and/or at least one mutation, deletion and/or insertion in LKB1.

### SUMMARY OF THE INVENTION

In one embodiment of the present invention methods are provided for treating a mammal with cancer comprising administering a therapeutically effective amount of at least one MEK inhibitor and at least one mTOR inhibitor to said mammal, wherein said mammal has at least one mutation in at least one Ras protein or gene encoding at least one Ras protein and wherein said mammal has at least one mutation, deletion or insertion in LKB1/STK11.

In one embodiment of the present invention methods are provided for treating a human with cancer comprising:
detecting at least one mutation in a Ras protein or a gene encoding at least one
Ras protein from a sample from said human;
detecting the present or absence of at least one mutation, deletion or insertion in LKB1 from a sample from said human; and
treating said human with a pharmaceutical composition comprising at least one MEK inhibitor comprising a compound of Structure (I):

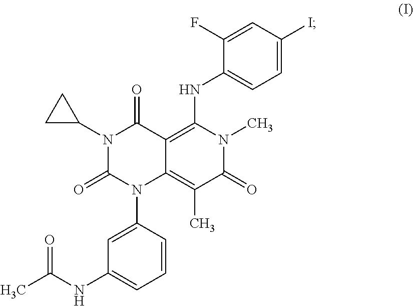 [SMILES: CC(=O)Nc1cccc(-n2c(=O)n(C3CC3)c(=O)c3c(Nc4ccc(I)cc4F)n(C)c(=O)c(C)c32)c1]

or a pharmaceutically acceptable salt or solvate thereof if at least one mutation in at least Ras protein or a gene encoding at least one Ras protein is detected and at least one mutation, deletion or insertion in LKB1 from a sample from said human is not detected.

In one embodiment of the present invention, methods are provided for treating a human with non-small cell lung carcinoma (NSCLC) wherein said human has at least one mutation in at least one Ras protein or gene encoding at least one Ras protein comprising administering to said human a therapeutically effective amount of a pharmaceutical composition comprising at least one MEK inhibitor comprising a compound of Structure (I):

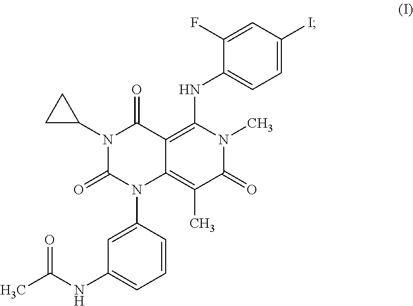 [SMILES: CC(=O)Nc1cccc(-n2c(=O)n(C3CC3)c(=O)c3c(Nc4ccc(I)cc4F)n(C)c(=O)c(C)c32)c1]

or a pharmaceutically acceptable salt or solvate thereof and at least one mTOR inhibitor.

### DETAILED DESCRIPTION OF THE INVENTION

In one embodiment of the present invention methods are provided for treating a mammal with cancer comprising administering a therapeutically effective amount of at least one MEK inhibitor and at least one mTOR inhibitor to said mammal, wherein said mammal has at least one mutation in at least one Ras protein or gene encoding at least one Ras protein and wherein said mammal has at least one mutation, deletion or insertion in LKB1/STK11.

In one embodiment of the present invention methods are provided for treating a mammal in need thereof with a therapeutically effective amount of at least one MEK inhibitor and at least one mTOR inhibitor comprising:
determining if said mammal has at least one mutation in at least one Ras protein or gene encoding at least one Ras protein and at least one mutation, deletion and/or insertion in LKB1; and
administering a therapeutically effective amount of at least one MEK inhibitor and at least one mTOR inhibitor to said mammal if said mammal has at least one mutation in at least one Ras protein or gene encoding at least one Ras protein and at least one mutation, deletion and/or insertion in LKB1.

In one embodiment of the present invention methods are provided for treating a mammal for cancer comprising the steps of:
obtaining at least one first sample from said mammal;
detecting at least one mutation in at least one Ras protein or gene encoding at least one Ras protein in said at least one first sample from said mammal, optionally obtaining at least one second sample from said mammal;
detecting at least one LKB1 mutation, deletion and/or insertion from at least one said first sample or at least one said optional second sample from said mammal, and
treating said mammal with a therapeutically effective amount of at least one MEK inhibitor and at least one mTOR inhibitor if at least one Ras mutation and at least one LKB1 mutation, deletion and/or insertion is detected in said first and/or second sample.

Methods are also provided for administering a therapeutically effective amount of at least one MEK inhibitor and at least one mTOR inhibitor to a mammal in need thereof comprising:
determining if said mammal has at least one mutation in at least one Ras protein or gene encoding at least one Ras protein and at least one mutation, deletion and/or insertion in LKB1; and
administering a therapeutically effective amount of at least one MEK inhibitor and at least one mTOR inhibitor to said mammal if said mammal has at least one mutation in at least one Ras protein or gene encoding at least one Ras protein and at least one mutation, deletion and/or insertion in LKB1.

In one aspect of the present invention the mammal is human.

In one aspect, at least one mutation in at least one Ras protein or gene encoding at least one Ras protein is in K-ras, N-ras or H-ras. Ras mutation in at least one gene encoding at least one Ras protein is in exon 2 and/or 3. In some instances, a gene encoding at least one Ras protein has a mutation in at least one of ras codon selected from: codon 12, 13, 14, 60, 61, 74, 76 and 146. In some aspects the Ras protein has a mutation selected from G12S, G12V, G12D, G12A, G12C, G12R, G12F, G13C, G13A, G13D, G13R, V14I, G60E, Q61H, Q61K, Q61R, T74P, E76G, E76K, E76Q, and A146T.

The wild type protein sequences for K-Ras, N-Ras, and H-Ras are known in the art and can be obtained from various databases including SwisProt database UniProtKB/Swiss-Prot: UniProtKB No. P01116 (K-ras); UniProtKB No. P01111 (N-ras), and P01112 (H-Ras), respectively. Also see Shimizu, et al., Proc. Natl. Acad. Sci. (U.S.A.), 80 (1983), pp. 2112-2116; Bos, Mutation research, Reviews in Genetic Toxicology 195 (30:255-271 (1988); and Fasano, et al., Mol. Cell. Biol., 4 (1984), pp. 1695-1705.

In another embodiment, the mammal has at least one missense mutation in LKB1 selected from: 581A>T causing amino acid change D194V; 842C>T causing amino acid change P281L; 595G>C causing amino acid change E199Q; 1062C>G causing amino acid change F354L; 521A>G causing amino acid change H174R; 526G>T causing amino acid change D176Y; 580G>T causing amino acid change D194Y; 580G>A causing amino acid change D194N; 166G>T causing amino acid change G56W; 167G>T causing amino acid change G56V; 587G>T causing amino acid change G196Y; 232A>G causing amino acid change K78E; 724G>C causing amino acid change G242R; 725G>T causing amino acid change G242V; 709G>T causing amino acid change D237Y; 910C>G causing amino acid change R304G; 829G>T causing amino acid change D277Y; 923G>T causing amino acid change W308L; 854T>A causing amino acid change L285Q; 1225C>T causing amino acid change R409W; 256C>G causing amino acid change R86G; 1062C>G causing amino acid change F354L; 816C>T causing amino acid change Y272Y; 487G>T causing amino acid change G163C; 368A>G causing amino acid change Q123R and/or 1276C>T causing amino acid change R426W.

In another embodiment, the mammal has at least one nonsense mutation in LKB1 selected from: 109C>T causing amino acid change Q37X; 508C>T causing amino acid change Q170X; 206C>A causing amino acid change S69X; 358G>T causing amino acid change E120X; 180C>G causing amino acid change Y60X; 180C>A causing amino acid change Y60X; 595G>T causing amino acid change E199X; 409C>T causing amino acid change Q137X; 493G>T causing amino acid change E165X; 571A>T causing amino acid change K191X; 658C>T causing amino acid change Q220X; 193G>T causing amino acid change E65X; 130A>T causing amino acid change K44X; 630C>A causing amino acid change C210X; 667G>T causing amino acid change E223X; 208G>T causing amino acid change E70X; 996G>A causing amino acid change W332X; 949G>T causing amino acid change E317X; 996G>A causing amino acid change W332X; 658C>T causing amino acid change Q220X and/or 475C>T causing amino acid change Q159X.

In another embodiment, the mammal has at least one deletion, insertion, substitution or complex mutation in LKB1 selected from: 120_{—}130del11; 153delG; 126_{—}149del24; 291_{—}464del174; 291_{—}597del307; 465_{—}597del133; 842delC; 735_{—}862del128; 166_{—}178del13; 431delC; 579delC; 157delG; 810delG; 598_{—}13del22; 544_{—}546delCTG; 827delG; 169delG; 291_{—}378del88; 598delG; 842delC; 465_{—}862del1398; 633delG; 1302del1302; 379_{—}433del55; 128_{—}129delC; 142_{—}143delA; 180delC; 209delA; 227_{—}228delC; 47_{—}651del605; 153_{—}536del384; exon 2-3del; exon 2-3del; exon 2-3del; exon 2-4-del; 562_{—}563delG; exon 4del; exon 4del; exon 4del; exon 4del; 610_{—}623del14; 837delC; 464_{—}465del2GGinsTTTGCT; 75_{—}76del2&insT; 125_{—}127insGG; 584_{—}585insT; 704_{—}705insA; 152_{—}153insCT; 842_{—}843insC; 649_{—}650insG; 127_{—}128insGG; 979_{—}980insAG; 165_{—}166insT; exon 6del; 1039_{—}1040insG; 735-2A>T; 5982AT; 465-1G>A; 465-1G>T; 29′-2A>T; 921-1G>A; and/or 597+1G>T; 143_{—}144>T; 841_{—}842>T; and/or 271_{—}272GG>TT.

In another embodiment, the deletion, insertion or mutation of LKB1 is in the catalytic kinase domain. The deletion, insertion or mutation of LKB1 may be in codons 50-337. In one embodiment, a mutation, deletion or insertion in LKB1 causes a truncated protein. The wild type nucleotide and protein sequence for LKB1 is known in the art and can be obtained from Q15831 (STK11_HUMAN) UniProtKB/Swiss-Prot

In one aspect the cancer is a solid tumor cancer. Solid tumor cancer may be lung cancer including but not limited to, non-small cell lung carcinoma (NSCLC), adenocarcinoma, or pancreatic cancer.

In one embodiment, the MEK inhibitor comprises a compound of Structure (I):

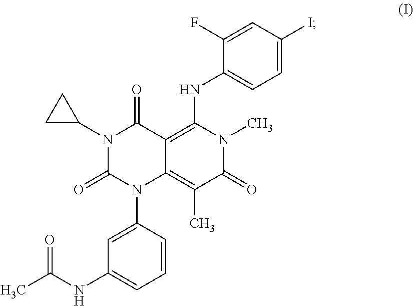 [SMILES: CC(=O)Nc1cccc(-n2c(=O)n(C3CC3)c(=O)c3c(Nc4ccc(I)cc4F)n(C)c(=O)c(C)c32)c1]

or a pharmaceutically acceptable salt or solvate thereof (hereinafter Compound A).

In one embodiment, the mTOR inhibitor is selected from rapamycin, rapalogs, everolimus, deforolimus, and temsirolimus. mTOR inhibitor may be referred to hereinafter as Compound B.

In one embodiment, the tumor cell also has at least one Braf mutation. Braf mutation include: R462I, I463S, G464V, G464E, G466A, G466E, G466V, G469A, G469E, D594V, F595L, G596R, L597V, L597R, T599I, V600E, V600D, V600K, V600R, T119S, and K601E.

In one embodiment, the mammal shows a complete response to a therapeutically effective amount of at least one MEK inhibitor and at least one mTOR inhibitor.

Also provided in the present invention are methods of treating a mammal for cancer comprising correlating the presence or absence of at least one mutation in a Ras protein or a gene encoding a Ras protein and the presence or absence of at least one mutation, deletion or insertion in LKB1 in at least one tumor cell from said mammal with an increased likelihood of response to treatment with at least one MEK inhibitor and at least one mTOR inhibitor.

In another embodiment, Structure (I), also referred to as N-{3-[3-cyclopropyl-5-(2-fluoro-4-iodo-phenylamino)-6,8-dimethyl-2,4,7-trioxo-3,4,6,7-tetrahydro-2H-pyrido[4,3-d]pyrimidin-1-yl]phenyl}acetamide, or a pharmaceutically acceptable salt or solvate thereof (hereinafter Compound A, or a pharmaceutically acceptable salt or solvate thereof) is in a sodium salt form. In another aspect, Compound A is in the form of a dimethyl sulfoxide solvate.

N-{3-[3-cyclopropyl-5-(2-fluoro-4-iodo-phenylamino)-6,8-dimethyl-2,4,7-trioxo-3,4,6,7-tetrahydro-2H-pyrido[4,3-d]pyrimidin-1-yl]phenyl}acetamide, or a pharmaceutically acceptable salt is a highly selective allosteric inhibitor of mitogen activated extracellular signal-regulated kinase 1 (MEK 1) and MEK 2. MEK proteins are a node in a certain extracellular signal-related kinase ERK pathway which is commonly hyper-activated In tumor cells. Oncogenic mutations in both B-raf and Ras signal through MEK1 and MEK2. In vitro, 80% of cell lines carrying activating mutations of B-Raf and 72% of Ras mutant cell lines were sensitive to N-{3-[3-cyclopropyl-5-(2-fluoro-4-iodo-phenylamino)-6,8-dimethyl-2,4,7-trioxo-3,4,6,7-tetrahydro-2H-pyrido[4,3-d]pyrimidin-1-yl]phenyl}acetamide, or a pharmaceutically acceptable salt in cell proliferation assays, and a majority (83% of hematopoietic cancers from acute or chronic myeloid leukemia (AML or CML, respectively) origins were also very sensitive.

Compound A is disclosed and claimed, along with pharmaceutically acceptable salts and solvates thereof, as being useful as an inhibitor of MEK activity, particularly in treatment of cancer, in International Application No. PCT/JP2005/011082, having an International filing date of Jun. 10, 2005; International Publication Number WO 2005/121142 and an International Publication date of Dec. 22, 2005, the entire disclosure of which is hereby incorporated by reference, Compound A is the compound of Example 4-1. Compound A can be prepared as described in International Application No. PCT/JP2005/011082. Compound A can be prepared as described in United States Patent Publication No. US 2006/0014768, Published Jan. 19, 2006, the entire disclosure of which is hereby incorporated by reference.

Suitably, Compound A is in the form of a dimethyl sulfoxide solvate. Suitably, Compound A is in the form of a sodium salt. Suitably, Compound A is in the form of a solvate selected from: hydrate, acetic acid, ethanol, nitromethane, chlorobenzene, 1-pentanci, isopropyl alcohol, ethylene glycol and 3-methyl-1-butanol. These solvates and salt forms can be prepared by one of skill in the art from, for example, the description in International Application No. PCT/JP2005/011082 or United States Patent Publication No. US 2006/0014768.

The compounds of the invention may contain one or more chiral atoms, or may otherwise be capable of existing as two enantiomers. Accordingly, the compounds of this invention include mixtures of enantiomers as well as purified enantiomers or enantiomerically enriched mixtures. Also, it is understood that all tautomers and mixtures of tautomers are included within the scope of Compound A, and pharmaceutically acceptable salts s thereof, and Compound B, and pharmaceutically acceptable salts thereof.

The compounds of the invention may form a solvate which is understood to be a complex of variable stoichiometry formed by a solute (in this invention, Compound A or a salt thereof and/or Compound B or a salt thereof) and a solvent. Such solvents for the purpose of the invention may not interfere with the biological activity of the solute. Examples of suitable solvents include, but are not limited to, water, methanol, dimethyl sulfoxide, ethanol and acetic acid. Suitably the solvent used is a pharmaceutically acceptable solvent. Examples of suitable pharmaceutically acceptable solvents include, without limitation, water, dimethyl sulfoxide, ethanol and acetic acid. Suitably the solvent used is water.

The pharmaceutically acceptable salts of the compounds of the invention are readily prepared by those of skill in the art.

Also, contemplated herein is a method of treating cancer using a combination of the invention where Compound A, or a pharmaceutically acceptable salt thereof, and/or Compound B or a pharmaceutically acceptable salt thereof are administered as pro-drugs. Pharmaceutically acceptable pro-drugs of the compounds of the invention are readily prepared by those of skill in the art.

By the term “treating” and grammatical variations thereof as used herein, is meant therapeutic therapy. In reference to a particular condition, treating means: (1) to ameliorate or prevent the condition of one or more of the biological manifestations of the condition, (2) to interfere with (a) one or more points in the biological cascade that leads to or is responsible for the condition or (b) one or more of the biological manifestations of the condition, (3) to alleviate one or more of the symptoms, effects or side effects associated with the condition or treatment thereof, or (4) to slow the progression of the condition or one or more of the biological manifestations of the condition. Prophylactic therapy is also contemplated thereby. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

As is understood in the art, the terms “complete remission,” “complete response” and “complete regression” mean the disappearance of all detectable signs and/or symptoms of cancer in response to treatment. As is also understood in the art detectable signs or symptoms of cancer can be defined based on the type and stage of cancer being treated. By way of example, “complete response” to treatment in a subject with NSCLC could be defined as no visible lung tumors observed with X-ray or CAT scan. In some instances, clinical response can be defined by RECIST 1.1 (Eisenhauer, 2009) as described below:

| Evaluation of target lesions | Evaluation of non-target lesions |
|---|---|
| Complete Response (CR): Disappearance of | While some non-target lesions may actually be |
| all target lesions. Any pathological lymph nodes | measurable, they need not be measured and |
| (whether target or non-target) must have | instead should be assessed only qualitatively at |
| reduction in short axis to <10 mm. | the time points specified in the protocol. |
| Partial Response (PR): At least a 30% | Complete Response (CR): Disappearance of |
| decrease in the sum of diameters of target | all non-target lesions and normalization of |
| lesions, taking as reference the baseline sum | tumor marker level. All lymph nodes must be |
| diameters. | non-pathological in size (<10 mm short axis). |
| Progressive Disease (PD): At least a 20% | Non-CR/Non-PD: Persistence of one or more |
| increase in the sum of diameters of target | non-target lesion(s) and/or maintenance of |
| lesions, taking as reference the smallest sum | tumor marker level above the normal limits. |
| on study (this includes the baseline sum if that | Progressive Disease (PD): |
| is the smallest on study). In addition to the | Unequivocal progression of existing non-target |
| relative increase of 20%, the sum must also | lesions. (Note: the appearance of one or more |
| demonstrate an absolute increase of at least 5 mm. | new lesions is also considered progression). |
| (Note: the appearance of one or more new | |
| lesions is also considered progression). | |
| Stable Disease (SD): Neither sufficient | |
| shrinkage to qualify for PR nor sufficient | |
| increase to qualify for PD, taking as reference | |
| the smallest sum diameters while on study. | |

Eisenhauer et al. European Journal of Cancer. 2009; 45: 228-247

“Clinical benefit response rate” as used herein for NSCLC means (CR+PR+SD≧mos). A positive clinical response can mean CR+PR after receiving treatment.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

By the term “combination” and grammatical variations thereof, as used herein is meant either simultaneous administration or any manner of separate sequential administration of a therapeutically effective amount of Compound A, or a pharmaceutically acceptable salt thereof, and Compound B or a pharmaceutically acceptable salt thereof. Preferably, if the administration is not simultaneous, the compounds are administered in a close time proximity to each other. Furthermore, it does not matter if the compounds are administered in the same dosage form, e.g. one compound may be administered topically and the other compound may be administered orally. Suitably, both compounds are administered orally.

By the term “combination kit” as used herein is meant the pharmaceutical composition or compositions that are used to administer Compound A, or a pharmaceutically acceptable salt thereof, and Compound B, or a pharmaceutically acceptable salt thereof, according to the invention. When both compounds are administered simultaneously, the combination kit can contain Compound A, or a pharmaceutically acceptable salt thereof, and Compound B, or a pharmaceutically acceptable salt thereof, in a single pharmaceutical composition, such as a tablet, or in separate pharmaceutical compositions. When the compounds are not administered simultaneously, the combination kit will contain Compound A, or a pharmaceutically acceptable salt thereof, and Compound B, or a pharmaceutically acceptable salt thereof, in separate pharmaceutical compositions. The combination kit can comprise Compound A, or a pharmaceutically acceptable salt thereof, and Compound B, or a pharmaceutically acceptable salt thereof, in separate pharmaceutical compositions in a single package or in separate pharmaceutical compositions in separate packages.

In one aspect there is provided a combination kit comprising the components:
Compound A, or a pharmaceutically acceptable salt thereof, in association with a pharmaceutically acceptable carrier; and
Compound B, or a pharmaceutically acceptable salt thereof, in association with a pharmaceutically acceptable carrier.

In one embodiment of the invention the combination kit comprises the following components:
Compound A, or a pharmaceutically acceptable salt thereof, in association with a pharmaceutically acceptable carrier; and
Compound B, or a pharmaceutically acceptable salt thereof, in association with a pharmaceutically acceptable carrier,
   wherein the components are provided in a form which is suitable for sequential, separate and/or simultaneous administration.

In one embodiment the combination kit comprises:
a first container comprising Compound A, or a pharmaceutically acceptable salt thereof, in association with a pharmaceutically acceptable carrier; and
a second container comprising Compound B, or a pharmaceutically acceptable salt thereof, in association with a pharmaceutically acceptable carrier, and a container means for containing said first and second containers.

The “combination kit” can also be provided by instruction, such as dosage and administration instructions. Such dosage and administration instructions can be of the kind that is provided to a doctor, for example by a drug product label, or they can be of the kind that is provided by a doctor, such as instructions to a patient.

As used herein the term “Compound A²” means —Compound A, or a pharmaceutically acceptable salt thereof—.

As used herein the term “Compound B²” means —Compound B, or a pharmaceutically acceptable salt thereof—.

Suitably the combinations of this invention are administered within a “specified period”.

By the term “specified period” and grammatical variations thereof, as used herein is meant the interval of time between the administration of one of Compound A² and Compound B² and the other of Compound A² and Compound B². Unless otherwise defined, the specified period can include simultaneous administration. Unless otherwise defined the specified period refers to administration of Compound A² and Compound B² during a single day.

Suitably, if the compounds are administered within a “specified period” and not administered simultaneously, they are both administered within about 24 hours of each other—in this case, the specified period will be about 24 hours; suitably they, will both be administered within about 12 hours of each other—in this case, the specified period will be about 12 hours; suitably they will both be administered within about 11 hours of each other—in this case, the specified period will be about 11 hours; suitably they will both be administered within about 10 hours of each other—in this case, the specified period will be about 10 hours; suitably they will both be administered within about 9 hours of each other—in this case, the specified period will be about 9 hours; suitably they will both be administered within about 8 hours of each other—in this case, the specified period will be about 8 hours; suitably they will both be administered within about 7 hours of each other—in this case, the specified period will be about 7 hours; suitably they will both be administered within about 6 hours of each other—in this case, the specified period will be about 6 hours; suitably they will both be administered within about 5 hours of each other—in this case, the specified period will be about 5 hours; suitably they will both be administered within about 4 hours of each other—in this case, the specified period will be about 4 hours; suitably they will both be administered within about 3 hours of each other—in this case, the specified period will be about 3 hours; suitably they will be administered within about 2 hours of each other—in this case, the specified period will be about 2 hours; suitably they will both be administered within about 1 hour of each other—in this case, the specified period will be about 1 hour. As used herein, the administration of Compound A² and Compound B² in less than about 45 minutes apart is considered simultaneous administration.

Suitably, when the combination of the invention is administered for a “specified period”, the compounds will be co-administered for a “duration of time”.

By the term “duration of time” and grammatical variations thereof, as used herein is meant that both compounds of the invention are administered for an indicated number of consecutive days. Unless otherwise defined, the number of consecutive days does not have to commence with the start of treatment or terminate with the end of treatment, it is only required that the number of consecutive days occur at some point during the course of treatment.

### Regarding “Specified Period” Administration:

Suitably, both compounds will be administered within a specified period for at least one day—in this case, the duration of time will be at least one day; suitably, during the course to treatment, both compounds will be administered within a specified period for at least 3 consecutive days—in this case, the duration of time will be at least 3 days; suitably, during the course to treatment, both compounds will be administered within a specified period for at least 5 consecutive days—in this case, the duration of time will be at least 5 days; suitably, during the course to treatment, both compounds will be administered within a specified period for at least 7 consecutive days—in this case, the duration of time will be at least 7 days; suitably, during the course to treatment, both compounds will be administered within a specified period for at least 14 consecutive days—in this case, the duration of time will be at least 14 days; suitably, during the course to treatment, both compounds will be administered within a specified period for at least 30 consecutive days—in this case, the duration of time will be at least 30 days.

Suitably, if the compounds are not administered during a “specified period”, they are administered sequentially. By the term “sequential administration”, and derivates thereof, as used herein is meant that one of Compound A² and Compound B² is administered once a day for two or more consecutive days and the other of Compound A² and Compound B² is subsequently administered once a day for two or more consecutive days. Also, contemplated herein is a drug holiday utilized between the sequential administration of one of Compound A² and Compound B² and the other of Compound A² and Compound B². As used herein, a drug holiday is a period of days after the sequential administration of one of Compound A² and Compound B² and before the administration of the other of Compound A² and Compound B² where neither Compound A² nor Compound B² is administered. Suitably the drug holiday will be a period of days selected from: 1 day, 2 days, 3 days, 4 days, 5 days, 6 days, 7 days, 8 days, 9 days, 10 days, 11 days, 12 days, 13 days and 14 days.

### Regarding Sequential Administration:

Suitably, one of Compound A² and Compound B² is administered for from 2 to 30 consecutive days, followed by an optional drug holiday, followed by administration of the other of Compound A² and Compound B² for from 2 to 30 consecutive days. Suitably, one of Compound A² and Compound B² is administered for from 2 to 21 consecutive days, followed by an optional drug holiday, followed by administration of the other of Compound A² and Compound B² for from 2 to 21 consecutive days. Suitably, one of Compound A² and Compound B² is administered for from 2 to 14 consecutive days, followed by a drug holiday of from 1 to 14 days, followed by administration of the other of Compound A² and Compound B² for from 2 to 14 consecutive days. Suitably, one of Compound A² and Compound B² is administered for from 3 to 7 consecutive days, followed by a drug holiday of from 3 to 10 days, followed by administration of the other of Compound A² and Compound B² for from 3 to 7 consecutive days.

Suitably, Compound B² will be administered first in the sequence, followed by an optional drug holiday, followed by administration of Compound A². Suitably, Compound B² is administered for from 3 to 21 consecutive days, followed by an optional drug holiday, followed by administration of Compound A² for from 3 to 21 consecutive days. Suitably, Compound B² is administered for from 3 to 21 consecutive days, followed by a drug holiday of from 1 to 14 days, followed by administration of Compound A² for from 3 to 21 consecutive days. Suitably, Compound B² is administered for from 3 to 21 consecutive days, followed by a drug holiday of from 3 to 14 days, followed by administration of Compound A² for from 3 to 21 consecutive days. Suitably, Compound B² is administered for 21 consecutive days, followed by an optional drug holiday, followed by administration of Compound A² for 14 consecutive days. Suitably, Compound B² is administered for 14 consecutive days, followed by a drug holiday of from 1 to 14 days, followed by administration of Compound A² for 14 consecutive days. Suitably, Compound B² is administered for 7 consecutive days, followed by a drug holiday of from 3 to 10 days, followed by administration of Compound A² for 7 consecutive days. Suitably, Compound B² is administered for 3 consecutive days, followed by a drug holiday of from 3 to 14 days, followed by administration of Compound A² for 7 consecutive days. Suitably, Compound B² is administered for 3 consecutive days, followed by a drug holiday of from 3 to 10 days, followed by administration of Compound A² for 3 consecutive days.

It is understood that a “specified period” administration and a “sequential” administration can be followed by repeat dosing or can be followed by an alternate dosing protocol, and a drug holiday may precede the repeat dosing or alternate dosing protocol.

Suitably, the amount of Compound A² administered as part of the combination according to the present invention will be an amount selected from about 0.125 mg to about 10 mg; suitably, the amount will be selected from about 0.25 mg to about 9 mg; suitably, the amount will be selected from about 0.25 mg to about 8 mg; suitably, the amount will be selected from about 0.5 mg to about 8 mg; suitably, the amount will be selected from about 0.5 mg to about 7 mg; suitably, the amount will be selected from about 1 mg to about 7 mg; suitably, the amount will be about 5 mg. Accordingly, the amount of Compound A administered as part of the combination according to the present invention will be an amount selected from about 0.125 mg to about 10 mg. For example, the amount of Compound A² administered as part of the combination according to the present invention can be 0.125 mg, 0.25 mg, 0.5 mg, 0.75 mg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg.

Suitably, the amount of Compound B² administered as part of the combination according to the present invention will be an amount selected from about 75 mg to about 1,000 mg; suitably, the amount will be selected from about 100 mg to about 900 mg; suitably, the amount will be selected from about 150 mg to about 850 mg; suitably, the amount will be selected from about 200 mg to about 800 mg; suitably, the amount will be selected from about 250 mg to about 750 mg; suitably, the amount will be selected from about 300 mg to about 6000 mg; suitably, the amount will be about 450 mg. Accordingly, the amount of Compound B² administered as part of the combination according to the present invention will be an amount selected from about 75 mg to about 1,000 mg. For example, the amount of Compound B² administered as part of the combination according to the present invention can be 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg or 1,000 mg.

As used herein, all amounts specified for Compound A² and Compound B² are indicated as the administered amount of free or unsalted and unsolvated compound per dose.

The method of the present invention may also be employed with other therapeutic methods of cancer treatment.

While it is possible that, for use in therapy, therapeutically effective amounts of the combinations of the present invention may be administered as the raw chemical, it is preferable to present the combinations as a pharmaceutical composition or compositions. Accordingly, the invention further provides pharmaceutical compositions, which include Compound A² and/or Compound B², and one or more pharmaceutically acceptable carriers. The combinations of the present invention are as described above. The carrier(s) must be acceptable in the sense of being compatible with the other ingredients of the formulation, capable of pharmaceutical formulation, and not deleterious to the recipient thereof. In accordance with another aspect of the invention there is also provided a process for the preparation of a pharmaceutical formulation including admixing Compound A² and/or Compound B² with one or more pharmaceutically acceptable carriers. As indicated above, such elements of the pharmaceutical combination utilized may be presented in separate pharmaceutical compositions or formulated together in one pharmaceutical formulation.

Pharmaceutical formulations may be presented in unit dose forms containing a predetermined amount of active ingredient per unit dose. As is known to those skilled in the art, the amount of active ingredient per dose will depend on the condition being treated, the route of administration and the age, weight and condition of the patient. Preferred unit dosage formulations are those containing a daily dose or sub-dose, or an appropriate fraction thereof, of an active ingredient. Furthermore, such pharmaceutical formulations may be prepared by any of the methods well known in the pharmacy art.

Compound A² and Compound B² may be administered by any appropriate route. Suitable routes include oral, rectal, nasal, topical (including buccal and sublingual), vaginal, and parenteral (including subcutaneous, intramuscular, intravenous, intradermal, intrathecal, and epidural). It will be appreciated that the preferred route may vary with, for example, the condition of the recipient of the combination and the cancer to be treated. It will also be appreciated that each of the agents administered may be administered by the same or different routes and that Compound A² and Compound B² may be compounded together in a pharmaceutical composition/formulation.

The compounds or combinations of the current invention are incorporated into convenient dosage forms such as capsules, tablets, or injectable preparations. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid. Liquid carriers include syrup, peanut oil, olive oil, saline, and water. Similarly, the carrier may include a prolonged release material, such as glyceryl monostearate or glyceryl distearate, alone or with a wax. The amount of solid carrier varies widely but, preferably, will be from about 25 mg to about 1 g per dosage unit. When a liquid carrier is used, the preparation will suitably be in the form of a syrup, elixir, emulsion, soft gelatin capsule, sterile injectable liquid such as an ampoule, or an aqueous or nonaqueous liquid suspension.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

It should be understood that in addition to the ingredients mentioned above, the formulations may include other agents conventional in the art having regard to the type of formulation in question, for example those suitable for oral administration may include flavoring agents.

As indicated, therapeutically effective amounts of the combinations of the invention (Compound A² in combination with Compound B²) are administered to a human. Typically, the therapeutically effective amount of the administered agents of the present invention will depend upon a number of factors including, for example, the age and weight of the subject, the precise condition requiring treatment, the severity of the condition, the nature of the formulation, and the route of administration. Ultimately, the therapeutically effective amount will be at the discretion of the attendant physician.

The combinations of the present invention are tested for efficacy, advantageous and synergistic properties according to known procedures. Suitably, the combinations of the invention are tested for efficacy, advantageous and synergistic properties generally according to the following combination cell proliferation assays. Cells are plated in 384-well plates at 500 cells/well in culture media appropriate for each cell type, supplemented with 10% FBS and 1% penicillin/streptomycin, and incubated overnight at 37° C., 5% CO₂. Cells are treated in a grid manner with dilution of Compound A² (20 dilutions, including no compound, of 2-fold dilutions starting from 1-20 μM depending of compound) from left to right on 384-well plate and also treated with Compound B² (20 dilutions, including no compound, of 2-fold dilutions starting from 1-20 μM depending of compound) from top to bottom on 384-well plate and incubated as above for a further 72 hours. In some instances compounds are added in a staggered manner and incubation time can be extended up to 7 days. Cell growth is measured using CellTiter-Glo® reagent according to the manufacturer's protocol and signals are read on a PerkinElmer EnVision™ reader set for luminescence mode with a 0.5-second read. Data are analyzed as described below.

Results are expressed as a percentage of the t=0 value and plotted against compound(s) concentration. The t=0 value is normalized to 100% and represents the number of cells present at the time of compound addition. The cellular response is determined for each compound and/or compound combination using a 4- or 6-parameter curve fit of cell viability against concentration using the IDBS XLfit plug-in for Microsoft Excel software and determining the concentration required for 50% inhibition of cell growth (gIC₅₀). Background correction is made by subtraction of values from wells containing no cells. For each drug combination a Combination Index (CI), Excess Over Highest Single Agent (EOHSA) and Excess Over Bliss (EOBliss) are calculated according to known methods such as described in Chou and Talalay (1984) Advances in Enzyme Regulation, 22, 37 to 55; and Berenbaum, M C (1981) Adv. Cancer Research, 35, 269-335.

Because the combinations of the present invention are active in the above assays they exhibit advantageous therapeutic utility in treating cancer. In some instances the cancer may be a solid tumor cancer. In other instances the cancer may be a liquid tumor such as, but not limited to, leukemia, myeloma, or lymphoma.

Suitably, the present invention relates to a method for treating or lessening the severity of a cancer selected from: brain (gliomas), glioblastomas, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumor of bone, thyroid,

Lymphoblastic T cell leukemia, Chronic myelogenous leukemia, Chronic lymphocytic leukemia, Hairy-cell leukemia, acute lymphoblastic leukemia, acute myelogenous leukemia, Chronic neutrophilic leukemia, Acute lymphoblastic T cell leukemia, Plasmacytoma, Immunoblastic large cell leukemia, Mantle cell leukemia, Multiple myeloma Megakaryoblastic leukemia, multiple myeloma, acute megakaryocytic leukemia, protyelocytic leukemia, Erythroleukemia,

malignant lymphoma, hodgkins lymphoma, non-hodgkins lymphoma, lymphoblastic T cell lymphoma, Burkitt's lymphoma, follicular lymphoma,

neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

Suitably, the present invention relates to a method for treating or lessening the severity of a cancer selected from: brain (gliomas), glioblastomas, Bannayan-Zonana syndrome, Cowden disease, Lhermitte-Duclos disease, breast, colon, head and neck, kidney, lung, liver, melanoma, ovarian, pancreatic, prostate, sarcoma and thyroid.

Suitably, the present invention relates to a method for treating or lessening the severity of a cancer selected from ovarian, breast, pancreatic and prostate.

Suitably, the invention relates to methods of treating lung cancer, including but not limited to, non-small cell; Non-small cell lung cancer; NSCLC; Adenocarcinoma—lung; and Squamous cell carcinoma.

Suitably the present invention relates to methods for treating or lessening the severity of a cancer selected from leukemia and myeloid malignancy.

Suitably the present invention relates to methods for treating or lessening the severity of epithelial cancer.

As used herein, the terms “cancer,” “neoplasm,” and “tumor,” are used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient. Tumors may be hematopoietic tumor, for example, tumors of blood cells or the like, meaning liquid tumors. Specific examples of clinical conditions based on such a tumor include leukemia such as chronic myelocytic leukemia or acute myelocytic leukemia; myeloma such as multiple myeloma; lymphoma and the like.

Typically, any anti-neoplastic agent that has activity versus a susceptible tumor being treated may be co-administered in the treatment of cancer in the present invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; receptor tyrosine kinase inhibitors; serine-threonine kinase inhibitors; non-receptor tyrosine kinase inhibitors; angiogenesis inhibitors, immunotherapeutic agents; proapoptotic agents; and cell cycle signalling inhibitors.

The present invention also provides methods for treating cancer comprising administering Compound A or pharmaceutically acceptable salt thereof with or without a Braf inhibitor, including, but not limited to, Compound B or a pharmaceutically acceptable salt thereof and another anti-neoplastic agent.

Examples of a further active ingredient or ingredients (anti-neoplastic agent) for use in combination or co-administered with Compound A or pharmaceutically acceptable salt thereof are chemotherapeutic agents.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the G₂/M phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 5β,20-epoxy-1,2α,4,7β,10β,13α-hexa-hydroxytax-11-en-9-one 4,10-diacetate 2-benzoate 13-ester with (2R,3S)—N-benzoyl-3-phenylisoserine; is a natural diterpene product isolated from the Pacific yew tree Taxus brevifolia and is commercially available as an injectable solution TAXOL®. It is a member of the taxane family of terpenes. It was first isolated in 1971 by Wani et al. J. Am. Chem., Soc., 93:2325. 1971), who characterized its structure by chemical and X-ray crystallographic methods. One mechanism for its activity relates to paclitaxel's capacity to bind tubulin, thereby inhibiting cancer cell growth. Schiff et al., Proc. Natl. Acad. Sci. USA, 77:1561-1565 (1980); Schiff et al., Nature, 277:665-667 (1979); Kumar, J. Biol. Chem. 256: 10435-10441 (1981). For a review of synthesis and anticancer activity of some paclitaxel derivatives see: D. G. I. Kingston et al., Studies in Organic Chemistry vol. 26, entitled “New trends in Natural Products Chemistry 1986”, Attaur-Rahman, P. W. Le Quesne, Eds. (Elsevier, Amsterdam, 1986) pp 219-235.

Paclitaxel has been approved for clinical use in the treatment of refractory ovarian cancer in the United States (Markman et al., Yale Journal of Biology and Medicine, 64:583, 1991; McGuire et al., Ann. Intern, Med., 111:273, 1989) and for the treatment of breast cancer (Holmes et al., J. Nat. Cancer Inst., 83:1797, 1991.) It is a potential candidate for treatment of neoplasms in the skin (Einzig et. al., Proc. Am. Soc. Clin. Oncol., 20:46) and head and neck carcinomas (Forastire et. al., Sem. Oncol., 20:56, 1990). The compound also shows potential for the treatment of polycystic kidney disease (Woo et. al., Nature, 368:750. 1994), lung cancer and malaria. Treatment of patients with paclitaxel results in bone marrow suppression (multiple cell lineages, Ignoff, R. J. et. al, Cancer Chemotherapy Pocket Guide, 1998) related to the duration of dosing above a threshold concentration (50 nM) (Kearns, C. M. et. al., Seminars in Oncology, 3(6) p. 16-23, 1995).

Docetaxel, (2R,3S)—N-carboxy-3-phenylisoserine,N-tert-butyl ester, 13-ester with 5β-20-epoxy-1,2α,4,7β,10β,13α-hexahydroxytax-11-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel q.v., prepared using a natural precursor, 10-deacetyl-baccatin Ill, extracted from the needle of the European Yew tree. The dose limiting toxicity of docetaxel is neutropenia.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although, it has possible indication as a second line therapy of various solid tumors, it is primarily indicated in the treatment of testicular cancer and various lymphomas including Hodgkin's Disease; and lymphocytic and histiocytic lymphomas. Myelosuppression is the dose limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin's and non-Hodgkin's malignant lymphomas. Alopecia and neurologic effects are the most common side effect of vincristine and to a lesser extent myelosupression and gastrointestinal mucositis effects occur.

Vinorelbine, 3′,4′-didehydro-4′-deoxy-C′-norvincaleukoblastine [R—(R*,R*)-2,3-dihydroxybutanedioate (1:2)(salt)], commercially available as an injectable solution of vinorelbine tartrate (NAVELBINE®), is a semisynthetic vinca alkaloid. Vinorelbine is indicated as a single agent or in combination with other chemotherapeutic agents, such as cisplatin, in the treatment of various solid tumors, particularly non-small cell lung, advanced breast, and hormone refractory prostate cancers. Myelosuppression is the most common dose limiting side effect of vinorelbine.

Platinum coordination complexes are non-phase specific anti-cancer agents, which are interactive with DNA. The platinum complexes enter tumor cells, undergo, aquation and form intra- and interstrand crosslinks with DNA causing adverse biological effects to the tumor. Examples of platinum coordination complexes include, but are not limited to, cisplatin and carboplatin.

Cisplatin, cis-diamminedichloroplatinum, is commercially available as PLATINOL® as an injectable solution. Cisplatin is primarily indicated in the treatment of metastatic testicular and ovarian cancer and advanced bladder cancer. The primary dose limiting side effects of cisplatin are nephrotoxicity, which may be controlled by hydration and diuresis, and ototoxicity.

Carboplatin, platinum, diammine [1,1-cyclobutane-dicarboxylate(2-)-O,O′], is commercially available as PARAPLATIN® as an injectable solution. Carboplatin is primarily indicated in the first and second line treatment of advanced ovarian carcinoma. Bone marrow suppression is the dose limiting toxicity of carboplatin.

Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN®. Cyclophosphamide is indicated as a single agent or in combination with other chemotherapeutic agents, in the treatment of malignant lymphomas, multiple myeloma, and leukemias. Alopecia, nausea, vomiting and leukopenia are the most common dose limiting side effects of cyclophosphamide.

Melphalan, 4-[bis(2-chloroethyl)amino]-L-phenylalanine, is commercially available as an injectable solution or tablets as ALKERAN®. Melphalan is indicated for the palliative treatment of multiple myeloma and non-resectable epithelial carcinoma of the ovary. Bone marrow suppression is the most common dose limiting side effect of melphalan.

Chlorambucil, 4-[bis(2-chloroethyl)amino]benzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin's disease. Bone marrow suppression is the most common dose limiting side effect of chlorambucil.

Busulfan, 1,4-butanediol dimethanesulfonate, is commercially available as MYLERAN® TABLETS. Busulfan is indicated for the palliative treatment of chronic myelogenous leukemia. Bone marrow suppression is the most common dose limiting side effects of busulfan.

Carmustine, 1,3-[bis(2-chloroethyl)-1-nitrosourea, is commercially available as single vials of lyophilized material as BiCNU®. Carmustine is indicated for the palliative treatment as a single agent or in combination with other agents for brain tumors, multiple myeloma, Hodgkin's disease, and non-Hodgkin's lymphomas. Delayed myelosuppression is the most common dose limiting side effects of carmustine.

Dacarbazine, 5-(3,3-dimethyl-1-triazeno)-imidazole-4-carboxamide, is commercially available as single vials of material as DTIC-Dome®. Dacarbazine is indicated for the treatment of metastatic malignant melanoma and in combination with other agents for the second line treatment of Hodgkin's Disease. Nausea, vomiting, and anorexia are the most common dose limiting side effects of dacarbazine.

Antibiotic anti-neoplastics are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthracyclins such as daunorubicin and doxorubicin; and bleomycins.

Dactinomycin, also know as Actinomycin D, is commercially available in injectable form as COSMEGEN®. Dactinomycin is indicated for the treatment of Wilm's tumor and rhabdomyosarcoma. Nausea, vomiting, and anorexia are the most common doss limiting side effects of dactinomycin.

Daunorubicin, (8S-cis-)-8-acetyl-10-[(3-amino-2,3,6-trideoxy-α-L-Iyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME® or as an injectable as CERUBIDINE®. Daunorubicin is indicated for remission induction in the treatment of acute nonlymphocytic leukemia and advanced HIV associated Kaposi's sarcoma. Myelosuppression is the most common dose limiting side effect of daunorubicin.

Doxorubicin, (8S,10S)-10-[(3-amino-2,3,6-trideoxy-α-L-Iyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component in the treatment of some solid tumors and lymphomas. Myelosuppression is the most common dose limiting side effect of doxorubicin.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas. Pulmonary and cutaneous toxicities are the most common dose limiting side effects of bleomycin.

Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Etoposide is indicated as a single agent or in combination with other chemotherapy agents in the treatment of testicular and non-small cell lung cancers. Myelosuppression is the most common side effect of etoposide. The incidence of leucopenia tends to be more severe than thrombocytopenia.

Teniposide, 4′-demethyl-epipodophyllotoxin 9[4,6-0-(R)-thenylidene-β-D-glucopyranoside], is commercially available as an injectable solution as VUMON® and is commonly known as VM-26. Teniposide is indicated as a single agent or in combination with other chemotherapy agents in the treatment of acute leukemia in children. Myelosuppression is the most common dose limiting side effect of teniposide. Teniposide can induce both leucopenia and thrombocytopenia.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mercaptopurine, thioguanine, and gemcitabine.

5-fluorouracil, 5-fluoro-2,4-(1H,3H) pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents in the treatment of carcinomas of the breast, colon, rectum, stomach and pancreas. Myelosuppression and mucositis are dose limiting side effects of 5-fluorouracil. Other fluoropyrimidine analogs include 5-fluoro deoxyuridine (floxuridine) and 5-fluorodeoxyuridine monophosphate.

Cytarabine, 4-amino-1-β-D-arabinofuranosyl-2 (1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2′,2′-difluorodeoxycytidine (gemcitabine). Cytarabine induces leucopenia, thrombocytopenia, and mucositis.

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. Myelosuppression and gastrointestinal mucositis are expected side effects of mercaptopurine at high doses. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Myelosuppression, including leucopenia, thrombocytopenia, and anemia, is the most common dose limiting side effect of thioguanine administration. However, gastrointestinal side effects occur and can be dose limiting. Other purine analogs include pentostatin, erythrohydroxynonyladenine, fludarabine phosphate, and cladribine.

Gemcitabine, 2′-deoxy-2′,2′-difluorocytidine monohydrochloride (β-isomer), is commercially available as GEMZAR®. Gemcitabine exhibits cell phase specificity at S-phase and by blocking progression of cells through the G1/S boundary. Gemcitabine is indicated in combination with cisplatin in the treatment of locally advanced non-small cell lung cancer and alone in the treatment of locally advanced pancreatic cancer. Myelosuppression, including leucopenia, thrombocytopenia, and anemia, is the most common dose limiting side effect of gemcitabine administration.

Methotrexate, N-[4[[(2,4-diamino-6-pteridinyl) methyl]methylamino]benzoy]R-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin's lymphoma, and carcinomas of the breast, head, neck, ovary and bladder. Myelosuppression (leucopenia, thrombocytopenia, and anemia) and mucositis are expected side effect of methotrexate administration.

Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (4S)-4,11-diethyl-4-hydroxy-9-[(4-piperidinopiperidino) carbonyloxy]-1H-pyrano[3′,4′,6,7]indolizino[1,2-b]quinoline-3,14(4H,12H)-dione hydrochloride, is commercially available as the injectable solution CAMPTOSAR®.

Irinotecan is a derivative of camptothecin which binds, along with its active metabolite SN-38, to the topoisomerase I—DNA complex. It is believed that cytotoxicity occurs as a result of irreparable double strand breaks caused by interaction of the topoisomerase I:DNA:irintecan or SN-38 ternary complex with replication enzymes. Irinotecan is indicated for treatment of metastatic cancer of the colon or rectum. The dose limiting side effects of irinotecan HCl are myelosuppression, including neutropenia, and GI effects, including diarrhea.

Topotecan HCl, (S)-10-[(dimethylamino)methyl]-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN®. Topotecan is a derivative of camptothecin which binds to the topoisomerase I—DNA complex and prevents religation of singles strand breaks caused by Topoisomerase I in response to torsional strain of the DNA molecule. Topotecan is indicated for second line treatment of metastatic carcinoma of the ovary and small cell lung cancer. The dose limiting side effect of topotecan HCl is myelosuppression, primarily neutropenia.

Pazopanib which commercially available as VOTRIENT® is a tyrosine kinase inhibitor (TKI). Pazopanib is presented as the hydrochloride salt, with the chemical name 5-[[4-[(2,3-dimethyl-2H-indazol-6-yl)methylamino]-2-pyrimidinyl]amino]-2-methylbenzenesulfonamide monohydrochloride. Pazoponib is approved for treatment of patients with advanced renal cell carcinoma.

Rituximab is a chimeric monoclonal antibody which is sold as RITUXAN® and MABTHERA®. Rituximab binds to CD20 on B cells and causes cell apoptosis. Rituximab is administered intravenously and is approved for treatment of rheumatoid arthritis and B-cell non-Hodgkin's lymphoma.

Ofatumumab is a fully human monoclonal antibody which is sold as ARZERRA®. Ofatumumab binds to CD20 on B cells and is used to treat chronic lymphocytic leukemia (CLL; a type of cancer of the white blood cells) in adults who are refractory to treatment with fludarabine (Fludara) and alemtuzumab (Campath).

mTOR inhibitors include but are not limited to rapamycin (FK506) and rapalogs, RAD001 or everolimus (Afinitor), CCl-779 or temsirolimus, AP23573, AZD8055, WYE-354, WYE-600, WYE-687 and Pp121.

Everolimus is sold as Afinitor® by Novartis and is the 40-O-(2-hydroxyethyl) derivative of sirolimus and works similarly to sirolimus as an mTOR (mammalian target of rapamycin) inhibitor. It is currently used as an immunosuppressant to prevent rejection of organ transplants and treatment of renal cell cancer. Much research has also been conducted on everolimus and other mTOR inhibitors for use in a number of cancers. It has the following chemical structure (formula II) and chemical name:

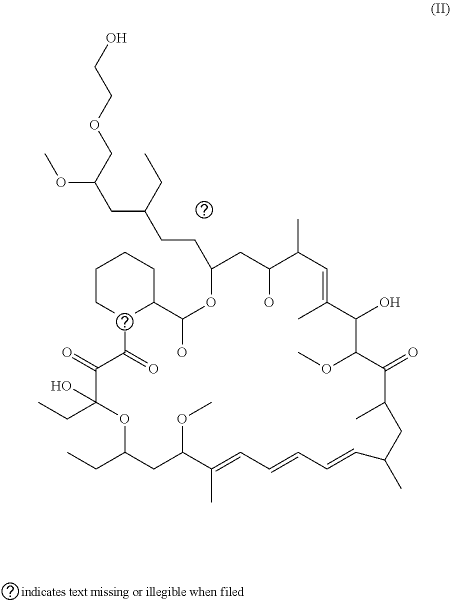 [SMILES: CCC(CCC1CC(O)C(C)/C=C(\C)C(O)C(OC)C(=O)C(C)CC(C)/C=C/C=C/C=C(\C)C(OC)CC(CC)OC(O)(CC)C(=O)C(=O)C2CCCCC2C(O)O1)CC(COCCO)OC]

dihydroxy-12-[(2R)-1-[(1S,3R,4R)-4-(2-hydroxyethoxy)-3-methoxycyclohexyl]propan-2-yl]-19,30-dimethoxy-15,17,21,23,29,35-hexamethyl-11,36-dioxa-4-azatricyclo[30.3.1.0^{4.9}]hexatriaconta-16,24,26,28-tetraene-2,3,10,14,20-pentone

Bexarotene is sold as Targretin® and is a member of a subclass of retinoids that selectively activate retinoid X receptors (RXRs). These retinoid receptors have biologic activity distinct from that of retinoic acid receptors (RARs). The chemical name is 4-[1-(5,6,7,8-tetrahydro-3,5,5,8,8-pentamethyl-2-naphthalenyl)ethenyl] benzoic acid. Bexarotene is used to treat cutaneous T-cell lymphoma (CTCL, a type of skin cancer) in people whose disease could not be treated successfully with at least one other medication.

Sorafenib marketed as Nexavar® is in a class of medications called multikinase inhibitors. Its chemical name is 4-[4-[[4-chloro-3-(trifluoromethyl)phenyl]carbamoylamino] phenoxy]-N-methyl-pyridine-2-carboxamide. Sorafenib is used to treat advanced renal cell carcinoma (a type of cancer that begins in the kidneys). Sorafenib is also used to treat unresectable hepatocellular carcinoma (a type of liver cancer that cannot be treated with surgery).

Suitably, the present invention relates to a method of treating or lessening the severity of a cancer that is either wild type or mutant for Raf and either wild type or mutant for PI3K/Pten. This includes patients wild type for both or either Raf and PI3K/PTEN while also being Ras mutant.

The term “wild type” as is understood in the art refers to a polypeptide or polynucleotide sequence that occurs in a native population without genetic modification. As is also understood in the art, a “mutant” includes a polypeptide or polynucleotide sequence having at least one modification to an amino acid or nucleic acid compared to the corresponding amino acid or nucleic acid found in a wild type polypeptide or polynucleotide, respectively. Included in the term mutant is Single Nucleotide Polymorphism (SNP) where a single base pair distinction exists in the sequence of a nucleic acid strand compared to the most prevalently found (wild type) nucleic acid strand.

As used herein, “genotyping” a cell including a tumor cell from a subject (or DNA or other biological sample) for a mutation or a polymorphic allele of a gene(s) means detecting which allelic or polymorphic form(s) and/or wild type or somatically mutated form(s) of the gene(s) or gene expression products (e.g., hnRNA, mRNA or protein) are present or absent in a subject (or a sample). Related RNA or protein expressed from such gene may also be used to detect polymorphic variation. For purposes of the present invention, “genotyping” includes the determination of somatic as well as genotypic mutations from a sample. As used herein, an allele may be ‘detected’ when other possible allelic variants have been ruled out; e.g., where a specified nucleic acid position is found to be neither adenine (A), thymine (T) or cytosine (C), it can be concluded that guanine (G) is present at that position (i.e., G is ‘detected’ or ‘diagnosed’ in a subject). Sequence variations may be detected directly (by, e.g. sequencing, for example, EST sequencing or partial or full genome sequencing) or indirectly (e.g., by restriction fragment length polymorphism analysis, or detection of the hybridization of a probe of known sequence, or reference strand conformation polymorphism), or by using other known methods.

The sequence of any nucleic acid including a gene or PCR product or a fragment or portion thereof may be sequenced by any method known in the art (e.g., chemical sequencing or enzymatic sequencing). “Chemical sequencing” of DNA may denote methods such as that of Maxam and Gilbert (1977) (Proc. Natl. Acad. Sci. USA 74:560), in which DNA is randomly cleaved using individual base-specific reactions. “Enzymatic sequencing” of DNA may denote methods such as that of Sanger (Sanger, et al., (1977) Proc. Natl. Acad. Sci. USA 74:5463).

Conventional molecular biology, microbiology, and recombinant DNA techniques including sequencing techniques are well known among those skilled in the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook, et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994

The Peptide Nucleic Acid (PNA) affinity assay is a derivative of traditional hybridization assays (Nielsen et al., Science 254:1497-1500 (1991); Egholm et al., J. Am. Chem. Soc. 114:1895-1897 (1992); James et al., Protein Science 3:1347-1350 (1994)). PNAs are structural DNA mimics that follow Watson-Crick base pairing rules, and are used in standard DNA hybridization assays. PNAs display greater specificity in hybridization assays because a PNA/DNA mismatch is more destabilizing than a DNA/DNA mismatch and complementary PNA/DNA strands form stronger bonds than complementary DNA/DNA strands.

DNA microarrays have been developed to detect genetic variations and polymorphisms (Taton et al., Science 289:1757-60, 2000; Lockhart et al., Nature 405:827-836 (2000); Gerhold et al., Trends in Biochemical Sciences 24:168-73 (1999); Wallace, R. W., Molecular Medicine Today 3:384-89 (1997); Blanchard and Hood, Nature Biotechnology 149:1649 (1996)). DNA microarrays are fabricated by high-speed robotics, on glass or nylon substrates, and contain DNA fragments with known identities (“the probe”). The microarrays are used for matching known and unknown DNA fragments (“the target”) based on traditional base-pairing rules.

The term “at least one mutation” in a polypeptide or a gene encoding a polypeptide and grammatical variations thereof means a polypeptide or gene encoding a polypeptide having one or more allelic variants, splice variants, derivative variants, substitution variants, deletion variants, truncation variants, and/or insertion variants, fusion polypeptides, orthologs, and/or interspecies homologs. By way of example, at least one mutation of a Ras protein would include a Ras protein in which part of all of the sequence of a polypeptide or gene encoding the Ras protein is absent or not expressed in the cell for at least one Ras protein produced in the cell. For example, a Ras protein may be produced by a cell in a truncated form and the sequence of the truncated form may be wild type over the sequence of the truncate. A deletion may mean the absence of all or part of a gene or protein encoded by a gene. Additionally, some of a protein expressed in or encoded by a cell may be mutated while other copies of the same protein produced in the same cell may be wild type. By way of another example a mutation in a Ras protein would include a Ras protein having one or more amino acid differences in its amino acid sequence compared with wild type of the same Ras protein. By way of another example, a mutation LKB1 includes, but is not limited to, an LKB1 having at least one amino acid difference compared to wild type LKB1. Mutation may be somatic or germline. Mutations in a polypeptide, including, but not limited to, LKB1, can lead to expression of truncated protein.

As used herein “genetic abnormality” is meant a deletion, substitution, addition, translocation, amplification and the like relative to the normal native nucleic acid content of a cell of a subject.

The terms “polypeptide” and “protein” are used interchangeably and are used herein as a generic term to refer to native protein, fragments, peptides, or analogs of a polypeptide sequence. Hence, native protein, fragments, and analogs are species of the polypeptide genus.

The terminology “X#Y” in the context of a mutation in a polypeptide sequence is art-recognized, where “#” indicates the location of the mutation in terms of the amino acid number of the polypeptide, “X” indicates the amino acid found at that position in the wild-type amino acid sequence, and “Y” indicates the mutant amino acid at that position. For example, the notation “G12S” with reference to the K-ras polypeptide indicates that there is a glycine at amino acid number 12 of the wild-type K-ras sequence, and that glycine is replaced with a serine in the mutant K-ras sequence.

The term “Ras protein” as used herein means any protein which is a member of the ras subfamily which is a subfamily of GTPases involved in cellular signaling. As is known in the art, activation of Ras causes cell growth, differentiation and survival. Ras proteins include, but are not limited to, H-ras, K-ras and N-ras.

As used herein “LKB1” is synonymous with Serine/Threonine Kinase 11 (STK11). The human LKB gene (official HUGO symbol, STK11) encodes a serine/threonine protein kinase that is defective in patients with Peutz-Jeghers syndrome (PJS). PJS is an autosomal dominantly inherited syndrome characterized by hamartomatous polyposis of the gastrointestinal tract and mucocutaneous pigmentation. To date, 145 different germline LKB1 mutations have been reported. The majority of the mutations lead to a truncated protein product. One mutational hotspot has been observed. A 1-bp deletion and a 1-bp insertion at the mononucleotide repeat (C6 repeat, c.837-c.842) between the codons 279-281 have been found in six and seven unrelated PJS families, respectively. However, these mutations account only for approximately 7% of all mutations identified in the PJS families (13/193). A review of the literature provides a total of 40 different somatic LKB1 mutations in 41 sporadic tumors and seven cancer cell lines. Mutations occur particularly in lung and colorectal cancer. Most of the somatic LKB1 mutations result in truncation of the protein. A mutational hotspot seems to be a C6 repeat accounting for 12.5% of all somatic mutations (6/48). These results are concordant with the germline mutation spectrum. However, the proportion of the missense mutations seems to be higher among the somatic mutations (45%) than among the germline mutations (21%), and only seven of the mutations are exactly the same in both of the mutation types. Hum Mutat 26(4), 291-297, 2005. Launonen. Human Mutation. 26(4), 291-297, 2005.

As used herein “gene encoding a Ras protein” means any part of a gene or polynucleotide encoding any Ras protein. Included within the meaning of this term are exons encoding Ras. Gene encoding Ras proteins include but are not limited to genes encoding part or all of H-ras, K-ras and N-ras.

The terms “mutant K-ras protein” and “mutant N-ras protein” and “K-ras mutation” and “N-ras mutation” refer to K-ras and N-ras proteins having at least one mutation, respectively. In certain embodiments, the Ras mutations include G12S, G12V, G12D, G12A, G12C, G12F, G12R, G13A, G13C, G13D, V14I, G60E, Q61H, Q61K, T74P, E76G, E76K, E76Q and A146T. Certain N-ras mutations include, but are not limited to G12S, G12V, G12D, G12A, G12C, G13A, G13D, G60E Q61H, and Q61K. Certain K-ras mutations can occur at positions 12, 13, 14, 61, and 76 and include, but are not limited, to, G12S, G12V, G12D, G12A, G12C, G12F, G12R, G13A, G13C, G13D, V141, G60E, Q61H, Q61K, T74P, E76G, E76K, E76Q and A146T. Ras protein mutation may occur at amino acid 12, 13, 14, 59, 60, 61, 76 and/or 146. Certain exemplary mutant K-ras and N-ras polypeptides include, but are not limited to, allelic variants, splice variants, derivative variants, substitution variants, deletion variants, and/or insertion variants, fusion polypeptides, orthologs, and interspecies homologs. In certain embodiments, a mutant K-ras and N-ras polypeptides includes additional residues at the C- or N-terminus, such as, but not limited to, leader sequence residues, targeting residues, amino terminal methionine residues, lysine residues, tag residues and/or fusion protein residues.

Additionally, mutant Ras polypeptides include polypeptides or gene encoding a polypeptide in which part of all of the polypeptide or gene encoding the polypeptide is deleted or absent from the cell. For example, a Ras protein may be produced by a cell in a truncated form. A deletion may mean the absence of all or part of a gene or protein encoded by a gene.

As used herein the term “amplification” and grammatical variations thereof refers to the presence of one or more extra gene copies in a chromosome complement. In certain embodiments a gene encoding a Ras protein may be amplified in a cell. Amplification of the HER2 gene has been correlated with certain types of cancer. Amplification of the HER2 gene has been found in human salivary gland and gastric tumor-derived cell lines, gastric and colon adenocarcinomas, and mammary gland adenocarcinomas. Semba et al., Proc. Natl. Acad. Sci. USA, 82:6497-6501 (1985); Yokota et al., Oncogene, 2:283-287 (1988); Zhou et al., Cancer Res., 47:6123-6125 (1987); King et al., Science, 229:974-976 (1985); Kraus et al., EMBO J., 6:605-610 (1987); van de Vijver et al., Mol. Cell. Biol., 7:2019-2023 (1987); Yamamoto et al., Nature, 319:230-234 (1986).

As used herein “overexpressed” and “overexpression” of a protein or polypeptide and grammatical variations thereof means that a given cell produces an increased number of a certain protein relative to a normal cell. By way of example, a ras protein may be overexpressed by a tumor cell relative to a non-tumor cell. Additionally, a mutant ras protein may be overexpressed compared to wild type ras protein in a cell. As is understood in the art, expression levels of a polypeptide in a cell can be normalized to a housekeeping gene such as actin. In some instances, a certain polypeptide may be underexpressed in a tumor cell compared with a non-tumor cell.

As used herein “nucleic acid necessary for expression of at least one gene product” refers to a nucleic acid sequence that encodes any portion of a gene and/or is operably linked to a nucleic acid encoding a gene product but does not necessarily comprise encoding sequence. By way of example, a nucleic acid sequence necessary for the expression of at least one gene product includes, but is not limited to, enhancers, promoters, regulatory sequences, start codons, stop codons, polyadenylation sequences, and/or encoding sequences. Expression levels of a polypeptide in a particular cell can be effected by, but not limited to, mutations, deletions and/or substitutions of various regulatory elements and/or non-encoding sequence in the cell genome.

The terms “mutant B-raf protein” refers to a B-raf polypeptide comprising at least one mutation. Certain exemplary mutant B-raf polypeptides include, but are not limited to, allelic variants, splice variants, derivative variants, substitution variants, deletion variants, and/or insertion variants, fusion polypeptides, orthologs, and interspecies homologs. In certain embodiments, a mutant B-raf polypeptide includes additional residues at the C- or N-terminus, such as, but not limited to, leader sequence residues, targeting residues, amino terminal methionine residues, lysine residues, tag residues and/or fusion protein residues. Certain B-raf mutants include but are not limited to BRAF having an amino acid substitution selected from the group consisting of R462I, I463S, G464V, G464E, G466A, G466E, G466V, G469A, G469E, D594V, F595L, G596R, L597V, L597R, T599I, V600E, V600D, V600K, V600R, T119S, and K601E. See, for example, FIG. 2 of Halilovic and Solvit (2008) Current Opinion in Pharmacology 8:419-26. BRAF encodes a RAS-regulated kinase that mediate cell growth and malignant transformation kinase pathway activation.

The term “polynucleotide” as referred to herein means a polymeric form of nucleotides of at least 10 bases in length, either ribonucleotides or deoxynucleotides or a modified form of either type of nucleotide. The term includes single and double stranded forms of DNA.

The term “oligonucleotide” referred to herein includes naturally occurring and modified nucleotides linked together by naturally occurring, and non-naturally occurring oligonucleotide linkages. Oligonucleotides are a polynucleotide subset generally comprising a length of 200 bases or fewer. Oligonucleotides for primers may be 10 to 60 bases in length and include 12, 13, 14, 15, 16, 17, 18, 19, or 20 to 40 bases in length. Oligonucleotides are usually single stranded, e.g. for probes, although oligonucleotides may be double stranded, e.g. for use in the construction of a gene mutant. Oligonucleotides can be either sense or antisense oligonucleotides.

An oligonucleotide probe, or probe, is a nucleic acid molecule which typically ranges in size from about 8 nucleotides to several hundred nucleotides in length. Such a molecule is typically used to identify a target nucleic acid sequence in a sample by hybridizing to such target nucleic acid sequence under stringent hybridization conditions. Hybridization conditions have been described in detail above.

PCR primers are also nucleic acid sequences, although PCR primers are typically oligonucleotides of fairly short length which are used in polymerase chain reactions. PCR primers and hybridization probes can readily be developed and produced by those of skill in the art, using sequence information from the target sequence. (See, for example, Sambrook et al., supra or Glick et al., supra).

As is known in the art, several primers are known for use in PCR for detecting Ras and Braf mutations. For example, primers for detecting mutations in Braf and K-ras are presented in several research articles and US patents including, but not limited to, Brose, et al. Cancer Research 62:6997-7000 (2002), Xu, et al. Cancer research 63:4561-4567 (2003), as well as U.S. Pat. No. 7,745,128, and several commercially available kits (see Dxs Diagnostic Innovations, Applied Biosystems, and Quest diagnostics).

Cancers that are either wild type or mutant for Ras/Raf and either wild type or mutant for PI3K/Pten are identified by known methods. For example, wild type or mutant Ras/Raf or PI3K/PTEN tumor cells can be identified by DNA amplification and sequencing techniques, DNA and RNA detection techniques, including, but not limited to Northern and Southern blot, respectively, and/or various biochip and array technologies. Wild type and mutant polypeptides can be detected by a variety of techniques including, but not limited to immunodiagnostic techniques such as ELISA, Western blot or immunocyto chemistry.

In one embodiment of the present invention, methods are provided for treating a mammal in need thereof with a therapeutically effective amount of at least one MEK inhibitor and at least one mTOR inhibitor comprising:
determining if said mammal has at least one mutation in at least one Ras protein or gene encoding at least one Ras protein and at least one mutation, deletion and/or insertion in LKB1; and
administering a therapeutically effective amount of at least one MEK inhibitor and at least one mTOR inhibitor to said mammal if said mammal has at least one mutation in at least one Ras protein or gene encoding at least one Ras protein and does not have at least one mutation, deletion and/or insertion in LKB1.

In one embodiment of the present invention, methods are provided for treating a human subject for cancer comprising the steps of:
obtaining at least one first sample from said subject;
detecting at least one mutation in at least one Ras protein or gene encoding at least one Ras protein in said at least one first sample from said subject;
optionally obtaining at least one second sample from said subject;
detecting at least one LKB1 mutation, deletion and/or insertion from at least one said first sample or at least one said optional second sample from said subject, and
treating said subject with a therapeutically effective amount of at least one MEK inhibitor and at least one mTOR inhibitor if at least one Ras mutation and at least one LKB1 mutation, deletion and/or insertion is detected in said first and/or second sample.

In one aspect, the first sample and said second sample are the same sample. In another aspect, the first sample is a tumor sample. In another aspect the first sample is a blood sample. In yet another aspect, the first sample and said second sample are different samples. The first sample may be a tumor sample and said second sample, may be a blood sample.

In one embodiment of the present invention, methods are provided for correlating the subject's increased or decreased likelihood of response to treatment with at least one MEK inhibitor and at least one mTOR inhibitor if said subject has at least one mutation in at least one Ras protein or gene encoding at least one Ras protein and/or at least one mutation, deletion or insertion in LKB1. In some instances, the LKB1 mutation leads to expression of a truncated LKB1 protein.

Suitably, the cancer may be a solid tumor cancer. In some instances, the cancer is non-small cell lung carcinoma (NSCLC) or pancreatic cancer.

Also provided herein, are methods of treating a human with cancer comprising
detecting at least one mutation in a Ras protein or a gene encoding at least one Ras protein from a sample from said human;
detecting the present or absence of at least one mutation, deletion or insertion in LKB1 from a sample from said human; and
treating said human with a pharmaceutical composition comprising at least one MEK inhibitor comprising a compound of Structure (I):

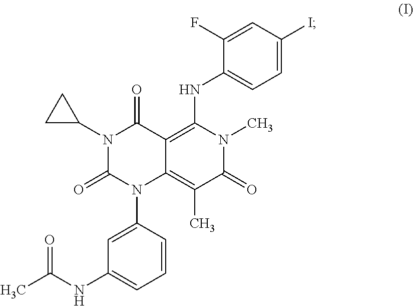 [SMILES: CC(=O)Nc1cccc(-n2c(=O)n(C3CC3)c(=O)c3c(Nc4ccc(I)cc4F)n(C)c(=O)c(C)c32)c1]

or a pharmaceutically acceptable salt or solvate thereof if at least one mutation in at least Ras protein or a gene encoding at least one Ras protein is detected and at least one mutation, deletion or insertion in LKB1 from a sample from said human is not detected.

In one aspect, the methods further comprise detecting the presence or absence of at least one BRG1 mutation.

Methods are provided for treating a human with NSCLC wherein said human has at least one mutation in at least one Ras protein or gene encoding at least one Ras protein comprising administering to said human a therapeutically effective amount of a pharmaceutical composition comprising at least one MEK inhibitor comprising a compound of Structure (I):

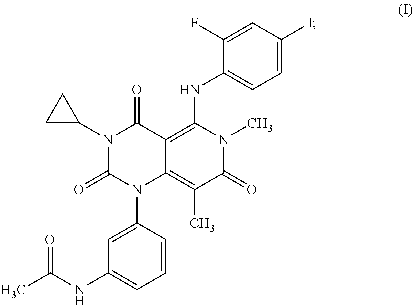 [SMILES: CC(=O)Nc1cccc(-n2c(=O)n(C3CC3)c(=O)c3c(Nc4ccc(I)cc4F)n(C)c(=O)c(C)c32)c1]

or a pharmaceutically acceptable salt or solvate thereof and at least one mTOR inhibitor. In one aspect the Ras mutation is selected from: G12S, G12V, G12D, G12A, G12C, G12R, G12F, G13C, G13A, G13D, V14I, G60E, Q61K, Q61H, T74P, E76G, E76K, E76Q and A146T.

Methods are provided for treating a human with NSCLC wherein said human has at least one mutation in at least one Ras protein or gene encoding at least one Ras protein comprising administering to said human a therapeutically effective amount of a pharmaceutical composition comprising at least one MEK inhibitor comprising a compound of Structure (I):

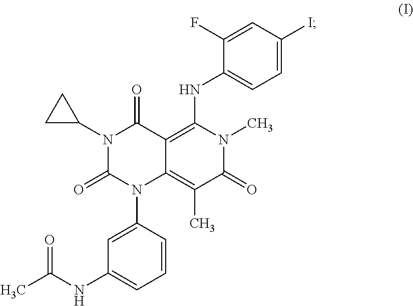 [SMILES: CC(=O)Nc1cccc(-n2c(=O)n(C3CC3)c(=O)c3c(Nc4ccc(I)cc4F)n(C)c(=O)c(C)c32)c1]

or a pharmaceutically acceptable salt or solvate thereof and at least one additional agent selected from the group of: docetaxel, PI3k/mTOR inhibitor, dasatinib, AKT inhibitor and everolimus. Also provided are methods of treating a human with NSCLC comprising correlating the human's likelihood to respond to treatment with Compound A in combination with docetaxel, PI3k/mTOR inhibitor, dasatinib, AKT inhibitor and/or an mTOR inhibitor, including, but not limited to, everolimus and the presence or absence of a RAS mutation in a tumor cell taken from said human.

PI3k/mTOR inhibitors are disclosed and claimed, along with pharmaceutically acceptable salts and solvates thereof, as being useful as inhibitors of PI3 kinases as well as methods of making and using PI3k/mTOR inhibitors particularly in treatment of cancer, in International Application No. PCT/US2008/063819 having an international filing date of May 16, 2008 and an International publication number of WO 2008/144463 and an International Publication date of Nov. 27, 2008, the entire disclosure of which is hereby incorporated by reference. A PI3k/mTOR inhibitor as used in the present invention is provided in example 345 of WO 2008/144463 and has the following structure:

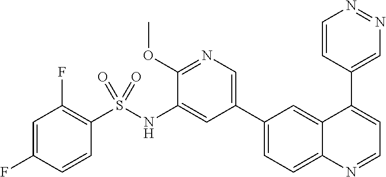 [SMILES: COc1ncc(-c2ccc3nccc(-c4ccnnc4)c3c2)cc1NS(=O)(=O)c1ccc(F)cc1F]

2,4-difluoro-N-{2-(methyloxy)-5-[4-(4-pyridazinyl)-6-quinolinyl]-3-pyridinyl}benzenesulfonamide.

AKT inhibitors are disclosed and claimed, along with pharmaceutically acceptable salts thereof, as being useful as an inhibitor of AKT activity, particularly in treatment of cancer, in International Application No. PCT/US2008/053269, having an International filing date of Feb. 7, 2008; International Publication Number WO 2008/098104 and an International Publication date of Aug. 14, 2008, the entire disclosure of which is hereby incorporated by reference. An AKT inhibitor as used in the present invention is described in example 96 and can be prepared as described in International Application No. PCT/US2008/053269 and is described by the following formula:

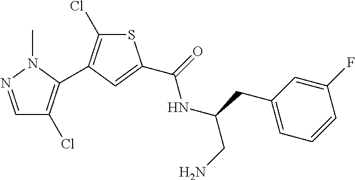 [SMILES: Cn1ncc(Cl)c1-c1cc(C(=O)N[C@H](CN)Cc2cccc(F)c2)sc1Cl]

N-{(1S)-2-amino-1-[(3-fluorophenyl)methyl]ethyl}-5-chloro-4-(4-chloro-1-methyl-1H-pyrazol-5-yl)-2-thiophenecarboxamide.

Suitably, N-{(1S)-2-amino-1-[(3-fluorophenyl)methyl]ethyl}-5-chloro-4-(4-chloro-1-methyl-1H-pyrazol-5-yl)-2-thiophenecarboxamide is in the form of a hydrochloride salt. The salt form can be prepared by one of skill in the art from the description in International Application No. PCT/US2010/022323, having an International filing date of Jan. 28, 2010; International Publication Number WO 2010/088331 and an International Publication date of Aug. 5, 2010, the entire disclosure of which is hereby incorporated by reference.

### EXAMPLES

The following examples are intended for illustration only and are not intended to limit the scope of the invention in any way.

### Example 1

Samples from humans who received at least one dose of a pharmaceutical composition comprising Structure I and at least one dose of an mTOR inhibitor will be tested for at least one mutation in a Ras protein or at least one mutation in a gene encoding a Ras protein mutation. Samples will also be tested for at least one mutation, deletion or insertion in LKB1. Data will be analyzed in order to correlate positive clinical response, either complete response (CR) and/or partial response (PR), to treatment with a pharmaceutical composition comprising Structure I and an mTOR inhibitor for patients with at least one mutation in a Ras protein or a gene encoding a Ras protein. Analyses will include samples from humans with NSCLC and pancreatic cancer. Additionally, data will be analyzed in order to correlate positive clinical response, either complete response (CR) and/or partial response (PR), to treatment with a pharmaceutical composition comprising Structure I and an mTOR inhibitor for patients with at least one mutation in a Ras protein or a gene encoding a Ras protein and at least one deletion, insertion or mutation in LKB1.

### RAS Mutation Kit:

RAS mutation testing will be performed in a CLIA certified lab (Genoptix Medical Laboratory) using a commercially available K-ras mutation kit provided by DxS Ltd. DxS K-RAS Mutation Kit is intended for the detection of the seven most common somatic mutations at codons 12 and 13 (12ALA, 12ASP, 12ARG, 12CYS, 12SER, 12VAL or 13ASP). The kit is for use on DNA samples and will provide a qualitative assessment of mutation status. This kit combines two technologies, ARMS and Scorpions, to detect mutations in real-time PCR reactions. Each assay is able to detect 1% mutation in a background of wild-type DNA.

Methods to Detect Mutations Deletions and/or Insertion in LKB1/STK11

Various methods are available to detect mutations, deletions and/or insertions in LKB1. These methods include, but are not limited to, the following:
DNA sequencing: Genomic DNA is subjected to a two step “boost/nest” PCR strategy, where the boost reaction generates a larger fragment to be used as a template for the nest reaction. The nest products are bidirectionally sequenced, base calling is performed and sequence tracing are visually inspected to confirm accurate variant detection by the base-calling software as described in Wingo et al., 2009. (Wingo et al., 2009. PLoS One. Somatic LKB1 mutations promote cervical cancer progression).
Multiple Ligation Probe Amplification (MLPA): LKB1 deletions or amplifications and duplications at exon or whole gene level can be searched using MLPA LKB1/STK11 kit available at vendors including but not limited to MRC-Holland in Wingo et al., 2009.
SURVEYOR digestion and High Pressure Liquid Chromatography (HPLC) analysis of PCR products: Specimens that show an altered pattern in the SURVEYOR tracings are purified and sequenced bidirectionally as described in Koivunen et al., 2008 (Koivunen et al., 2008. Br J cancer. Mutations in the LKB1 tumour suppressor are frequently detected in tumors from Caucasian but not Asian lung cancer patients).
Fine deletion mapping: Involves PCR amplification with radiolabelled polymorphic satellite markers followed by separation of a fraction of the PCR product on 8% urea-formamide-polyacrylamide gels and exposure to X-ray film. Allelic loss is scored by visually measuring reduction in the intensity in the tumor allele compared with the corresponding allele in the normal DNA as described in Sanchez-Cespedes et al, 2002 (Sanchez-Cespedes et al., 2002. Cancer Research. Inactivation of LKB1/STK11 is a common event in adenocarcinomas of the lung).
Screening for point mutation: Allele specific polymerase chain reaction for Exon 1, Exons 2-3, Exons 4-5, Exons 6-7, Exon 8 and Exon 9. Screening for point mutations may also involve cycle sequencing of Exons 1-9 as described in Sanchez-Cespedes et al, 2002.
Screening of homozygous deletions: PCR amplification of length polymorphisms as described in Sanchez-Cespedes et al, 2002.
Screening of partial deletions/insertions: Long-range PCR strategy by amplification of a 5-Kb fragment containing exons 2-8 as described in Sanchez-Cespedes et al, 2002.
Promoter hypermethylation analysis: Promoter methylation status is assayed by PCR using methylation-specific primers and nonmthylation primers as described in Sanchez-Cespedes et al, 2002.
   Phase II Evaluation of Structure I and Everolimus in Subjects with KRAS Mutant Non-Small Cell Lung Carcinoma (NSCLC)

The primary objective is to determine whether Structure I given in combination with everolimus is effective in KRAS-mutant NSCLC subjects. The null hypothesis is that the objective response rate (percentage of target subject population achieving CR or PR within 16 weeks of initiating combination treatment) to the combo treatment is not attractive (≦5%). The alternative hypothesis is that the response rate is of interest (≧20%) for further development. Symbolically, the hypotheses (H) are expressed as follows:

### H(0): Response Rate 55% (P0)

### H(1): Response Rate 20% (P1)

For final analysis, if the Bayesian posterior probability for the response rate >5% is large (decision rule, >0.98), or equivalent to observe at least 5 responses in 40 subjects (5/40) sufficient statistical evidence has been provided in favor of further development of Structure I and everolimus as a combo treatment in KRAS-mutant NSCLC subjects.

### Efficacy Analyses

Anti-tumor activities will be evaluated based on clinical evidence and the RECIST 1.1 criteria for solid tumors (Eisenhauer et al. European Journal of Cancer. 2009; 45: 228-247), which is summarized below:

| Evaluation of target lesions | Evaluation of non-target lesions |
|---|---|
| Complete Response (CR): Disappearance of | While some non-target lesions may actually be |
| all target lesions. Any pathological lymph nodes | measurable, they need not be measured and |
| (whether target or non-target) must have | instead should be assessed only qualitatively at |
| reduction in short axis to <10 mm. | the time points specified in the protocol. |
| Partial Response (PR): At least a 30% | Complete Response (CR): Disappearance of |
| decrease in the sum of diameters of target | all non-target lesions and normalization of |
| lesions, taking as reference the baseline sum | tumor marker level. All lymph nodes must be |
| diameters. | non-pathological in size (<10 mm short axis). |
| Progressive Disease (PD): At least a 20% | Non-CR/Non-PD: Persistence of one or more |
| increase in the sum of diameters of target | non-target lesion(s) and/or maintenance of |
| lesions, taking as reference the smallest sum | tumor marker level above the normal limits. |
| on study (this includes the baseline sum if that | Progressive Disease (PD): |
| is the smallest on study). In addition to the | Unequivocal progression of existing non-target |
| relative increase of 20%, the sum must also | lesions. (Note: the appearance of one or more |
| demonstrate an absolute increase of at least 5 mm. | new lesions is also considered progression). |
| (Note: the appearance of one or more new | |
| lesions is also considered progression). | |
| Stable Disease (SD): Neither sufficient | |
| shrinkage to qualify for PR nor sufficient | |
| increase to qualify for PD, taking as reference | |
| the smallest sum diameters while on study. | |

Eisenhauer et al. European Journal of Cancer. 2009; 45: 228-247

The objective response rate for Phase II NSCLC is defined as the percentage of subjects among all subjects who have enrolled and received at least one dose of a pharmaceutical composition comprising Structure I, who have a complete or a partial response within 16 weeks since the initiation of treatment. The clinical benefit response rate for Phase II NSCLC is defined as the percentage of subjects who have a complete or partial response, plus those who have stable disease for at least four months since first on study disease assessment. The duration of overall response is measured from the time measurement criteria are first met for complete response or partial response (whichever is first recorded) until the first date that progressive disease is objectively documented (taking as reference for progressive disease the smallest measurements recorded on study).

For Phase I, anti-tumor activities in pancreatic cancer, will be evaluated based on clinical evidence and response criteria. If the data warrant, the response data will be summarized by dose level.

For Phase II in NSCLC, statistical analyses will be performed to estimate the posterior distribution of the objective response rate using a Bayesian method. A non-informative Uniform (0, 1) prior will be used for the response rate. Based on the available data and the prior, a posterior distribution for the response rate will be determined. The mean of this distribution provides an estimate of the response rate with the corresponding 2.5th and 97.5th percentiles of the distribution providing a 95% Bayesian credible interval for the response rate. The posterior probability that the response rate >5% (P0), Pr(RR>5% |data) will be calculated and a graph of the posterior distribution for the response rate will be produced. The success criteria are based on the posterior distribution of the response rate at the final stage, where success (accept the new treatment) is defined i.e. Pr(RR>5%)>0.98. At least five responses are required in 40 subjects to meet these criteria. Clinical benefit response rate (CR+PR+SD≧4 mos) will be analyzed similar as the objective response rate. Posterior distribution of clinical benefit response rate will be constructed using a non-informative prior and the observed data.

Duration of overall response will be summarized descriptively in tabular and graphic formats. Correlation analysis will be conducted to explore any relationship between subjects's CA 19-9 level and tumor response based on RECIST 1.1 [Eisenhauer, 2009] for subjects with pancreatic cancer.

### Example 2

Targeting the activated RAS/RAF/MEK pathway in non small cell lung carcinoma (NSCLC) with the potent, selective MEK1/MEK2 small molecule inhibitor Compound A is an innovative approach to potentially improve the clinical outcome for patients with RAS-mutant tumors. This study assessed the in vitro susceptibility of RAS-mutant NSCLC cell lines to Compound A alone or in combination with PI3K/AKT/mTOR inhibitors, PI3Ki/mTOR inhibitor, AKT inhibitor, or everolimus (mTOR inhibitor) to identify markers of response/resistance and provide a scientific rationale for the clinical development of Compound A.

A panel of 24 non-small cell lung carcinoma (NSCLC) cell lines harboring RAS mutations was analyzed for genomic alterations, gene expression profiling and protein phosphorylation in MAPK and PI3K pathways. Inhibition of cell growth and induction of apoptosis were determined by CellTiter-Glo® and caspase 3/7 activation assays after 72 and 24 hours of treatment, respectively. Target and chemo agents, including Compound A, were tested alone and in combination with Compound A.
Targeted agents: erlotinib (EGFR inhibitor); everolimus (mTOR inhibitor), PI3K/mTOR inhibitor, PI3Kβ inhibitor, AKT inhibitor, PI3K inhibitor Chemo agents: docetaxel, pemetrexted, dasatinib

Baseline signaling pathways, genomic alterations and gene expression profiles were characterized.

Response Rates in RAS-Mutant NSCLC Lines by Compound a Alone or in Combination with Other Agents

Single Agent:

Compound A and docetaxel had cell apoptosis response rates of 25% and 42%, respectively; however each agent had a different sensitivity profile. PI3k/mTOR inhibitor potently inhibited cell growth, however resulted in little apoptosis. Dasatinib, everolimus, AKT inhibitor and erlotinib alone showed little or no activity.

Compound A in Combination with Other Agents:

The combination of Compound A plus docetaxel, PI3K/mTOR inhibitor, dasatinib or everolimus increased response rates compared with single agent. Compound A plus PI3k/mTOR inhibitor, AKT inhibitor or erlotinib showed benefit in cell growth inhibition. Compound A plus pemetrexed showed similar activity to the most active single agent.

Results of response rates in RAS mutant NSCLC cell lines for Compound A alone and with other agents are shown in Table 1.

TABLE 1
Response Rates in RAS-Mutant NSCLC Lines by
Compound A Alone or in Combination with Other Agents

| | | Response Rate | |
|---|---|---|---|
| | | (sensitivity/total lines tested) | |
| | | Ras^{mut} NCSLC Cell lines | |
| | | Cell growth | Cell Apoptosis |
| | Treatment (Target) | inhibition | (capsase 3/7) |
| Single Agent | Compound A | 38% (9/24) | 25% (6/24) |
| | Docataxel | 88% (21/24) | 42% (10/24) |
| | PI3k/mTOR inhibitor | 69% (9/13) | 6% (1/16) |
| | Pemtrexed (DHFR etc) | 38% (4/9) | NT |
| | Dasatinib (SRC) | 20% (2/10) | 0% (0/10) |
| | Everolimus (mTOR) | 8% (2/24) | 0% (0/24) |
| | AKT inhibitor | 6% (1/18) | 0% (0/9) |
| | Erlotinib (EGFR) | 0% (0/24) | 0% (0/24) |
| Combination | Compound A + | 96% (23/24) | 88% (21/24) |
| | Docataxel | | |
| | Compound A + PI3k/ | 100% (13/13) | 68% (11/16) |
| | mTOR inhibitor | | |
| | Compound A + | 70% (7/14) | 56% (5/9) |
| | Dasatinib (SRC) | | |
| | Compound A + | 88% (21/24) | 38% (9/24) |
| | Everolimus (mTOR) | | |
| | Compound A + AKT | 83% (15/18) | 33% (3/9) |
| | inhibitor | | |
| | Compound A + | 67% (6/9) | NT |
| | pemetrexed | | |
| | Compound A + Erlotinib | 56% (10/18) | 20% (2/10) |
| | (EGFR) | | |

Markers Associated with Compound A Response/Resistance were Identified.

LKB1 and BRG1 in NSCLC

Both LKB1 (9p13.2) and BRG1/SMARCA4 (19p13.3) genes are tumor suppressors and located on the same chromosome 19p which is frequently lost in lung cancer. LKB1 (STK11) regulates mTOR signaling, cellular energy metabolism and cell polarity by activating AMPK family. LKB1 loss heightens dependence on SRC and FAK signaling for cell adhesion and migration. Most somatic mutations of LKB1 involve nonsense or frameshift mutations leading to a truncated protein (Launonen, Human Mutation. 26(4):291-297 (2005); and Shaw et al., Science 310:1642-1646 (2005)). Low rate of somatic mutations (11%); LOH and homozygous deletion jointly occurred in ˜90% of the NSCLC. (Gill et al., Oncogene, 30:3784-3791 (2011)).

BRG1 (SMARCA4), a member of chromatin-remodeling SWI/SNF complex, plays a role in regulating gene transcription including cellular adhesion (eg, CD44, Strobeck, et al., J Biol. Chem. 276(12):9273-8. 2001; CDH1, Banine, I Cancer Res 65: 3542-3547 (2005)), and growth arrest (eg, cyclins A and E, Zhang, et al. Cell 101(1):78-89 (2000); CSF1, Liu, et al., Cell 106:309-318 (2001), and RB-mediated upregulating p21, Hendricks, et al., Mol. Cell. Biol. 24(1):362-376 (2004); and Bartlett, et al. Journal of Cellular Physiology. 226(8):1989-1997 (2011)). Direct physical interactions have been reported between the SWI/SNF complex and proteins that have important roles in cancer, such as RB, LKB1/STK11, βCATENIN, SMAD2, SMAD3, BRCA1, CMYC, MLL and CFOS. Most BRG1 alterations lead to truncated proteins. Loss of heterozygosity (LOH) at 19p and absence of the SMARCA4 protein have been reported in lung tumors.

Tumor suppressors, TP53, LKB1 (STK11), BRG1(SMARCA4), CDKN2A (p16) are frequently mutated in KRAS mutant NSCLC clinical samples. BRG1/LKB1/CDKN2A mutations are frequent and were found to be associated with resistance to Compound A as shown in various NSCLC cell lines in Table 2.

TABLE 2
Markers Associated with Compound A Response in RAS-mutant NSCLC Cells.

| | | Apoptosis Response | | | | | |
|---|---|---|---|---|---|---|---|
| | | | | Compound A + | Mutations | | |
| NSCLC | | Compound A | Docataxel | Docataxel | BRG1 | LKB1 | CDKN2A |
| Calu6 | KRAS^{Q61K} | ++ | ++ | ++++ | WT | WT | WT |
| H1792 | KRAS^{G12c} | ++ | +++ | ++++ | WT | WT | WT |
| H2291 | KRAS^{G12F} | ++ | − | ++ | WT | WT | WT |
| H2347 | KRAS^{Q61K} | ++ | − | ++ | WT | WT | WT |
| H2122 | KRAS^{G12c} | ++ | − | ++++ | WT | MUT | MUT |
| H727 | KRAS^{G12V} | ++ | − | ++ | WT | WT | WT |
| H358 | KRAS^{G12c} | − | ++ | +++ | WT | WT | WT |
| H1573 | KRAS^{G12A} | − | ++ | ++ | MUT | WT | WT |
| H460 | KRAS^{Q61H} | − | ++ | ++ | WT | MUT | MUT |
| H157 | KRAS^{G12R} | − | ++ | +++ | MUT | MUT | WT |
| HOP62 | KRAS^{G12c} | − | ++ | +++ | WT | WT | MUT |
| H23 | KRAS^{G12c} | − | +++ | ++++ | MUT | MUT | WT |
| A427 | KRAS^{G12D} | − | ++ | ++++ | MUT | MUT | WT |
| H2030 | KRAS^{G12c} | − | ++ | ++++ | MUT | MUT | WT |
| A549 | KRAS^{G12S} | − | − | ++ | MUT | MUT | MUT |
| H1299 | KRAS^{Q61H} | − | − | ++ | MUT | WT | WT |
| Calu1 | KRAS^{G12c} | − | − | ++ | WT | WT | WT |
| H1155 | KRAS^{Q61H} | − | − | ++ | WT | WT | WT |
| H1355 | KRAS^{G13C} | − | − | ++ | NULL | MUT | WT |
| SW1573 | KRAS^{G12C} | − | − | − | WT | WT | MUT |
| COR-L23 | KRAS^{G12V} | − | − | − | WT | WT | WT |
| SW900 | KRAS^{G12V} | − | − | ++ | WT | WT | MUT |
| H2009 | KRAS^{G12A} | − | − | +++ | WT | WT | WT |
| H441 | KRAS^{G12V} | − | − | − | WT | WT | WT |

“−”: lack of caspase 3/7 activation after 24 hours of compound treatment; “++, +++, ++++”: moderate, strong and very strong levels of caspase 3/7 activation after 24 hours of compound treatment; WT: wild type; MUT: mutant.

### CONCLUSIONS

These results provide a strong rationale for testing Compound A and these combinations in the clinic. BRG1/LKB1 mutations should be further investigated as markers for resistance/response to improve patient selection for these agents in RAS mutated NSCLC.

While the preferred embodiments of the invention are illustrated by the above, it is to be understood that the invention is not limited to the precise instructions herein disclosed and that the right to all modifications coming within the scope of the following claims is reserved.

1. A method of treating a mammal with cancer comprising administering a therapeutically effective amount of at least one MEK inhibitor and at least one mTOR inhibitor to said mammal, wherein said mammal has at least one mutation in at least one Ras protein or gene encoding at least one Ras protein and wherein said mammal has at least one mutation, deletion or insertion in LKB1/STK11.

2. The method of claim 1 wherein said at least one mutation in at least one Ras protein or gene encoding at least one Ras protein is in K-ras, N-ras or H-ras.

3. The method of any one of claim 1 or 2 wherein the mutation in at least one gene encoding at least one Ras protein is in exon 2 or 3.

4. The method of any one of claims 1 to 3 wherein, the gene encoding at least one Ras protein has a mutation in at least one of ras codon selected from: codon 12, 13, 14, 60, 61, 74, 76, and 146.

5. The method of any one of claims 1 to 4, wherein the Ras mutation is selected from: G12S, G12V, G12D, G12A, G12C, G12R, G12F, G13C, G13A, G13D, G13R, V14I, G60E, Q61H, Q61K, Q61R, T74P, E76G, E76K, E76Q and A146T.

6. The method of any one of claims 1 to 5 wherein the mammal has at least one missense mutation in LKB1 selected from: 581A>T causing amino acid change D194V; 842C>T causing amino acid change P281L; 595G>C causing amino acid change E199Q; 1062C>G causing amino acid change F354L; 521A>G causing amino acid change H174R; 526G>T causing amino acid change D176Y; 580G>T causing amino acid change D194Y; 580G>A causing amino acid change D194N; 166G>T causing amino acid change G56W; 167G>T causing amino acid change G56V; 587G>T causing amino acid change G196Y; 232A>G causing amino acid change K78E; 724G>C causing amino acid change G242R; 725G>T causing amino acid change G242V; 709G>T causing amino acid change D237Y; 910C>G causing amino acid change R304G; 829G>T causing amino acid change D277Y; 923G>T causing amino acid change W308L; 854T>A causing amino acid change L285Q; 1225C>T causing amino acid change R409W; 256C>G causing amino acid change R86G; 1062C>G causing amino acid change F354L; 816C>T causing amino acid change Y272Y; 487G>T causing amino acid change G163C; 368A>G causing amino acid change Q123R; and 1276C>T causing amino acid change R426W.

7. The method of any one of claims 1 to 6 wherein the mammal has at least one nonsense mutation in LKB1 selected from: 109C>T causing amino acid change Q37X; 508C>T causing amino acid change Q170X; 206C>A causing amino acid change S69X; 358G>T causing amino acid change E120X; 180C>G causing amino acid change Y60X; 180C>A causing amino acid change Y60X; 595G>T causing amino acid change E199X; 409C>T causing amino acid change Q137X; 493G>T causing amino acid change E165X; 571A>T causing amino acid change K191X; 658C>T causing amino acid change Q220X; 193G>T causing amino acid change E65X; 130A>T causing amino acid change K44X; 630C>A causing amino acid change C210X; 667G>T causing amino acid change E223X; 208G>T causing amino acid change E70X; 996G>A causing amino acid change W332X; 949G>T causing amino acid change E317X; 996G>A causing amino acid change W332X; 658C>T causing amino acid change Q220X; and 475C>T causing amino acid change Q159X.

8. The method of any one of claims 1 to 7 wherein said mammal has at least one deletion, insertion, substitution or complex mutation in LKB1 selected from: 120_{—}130del11; 153delG; 126_{—}149del24; 291464del174; 291_{—}597del307; 465_{—}597del133; 842delC; 735_{—}862del128; 166_{—}178del13; 431delC; 579delC; 157delG; 810delG; 598_{—}13del22; 544_{—}546delCTG; 827delG; 169delG; 291_{—}378del88; 598delG; 842delC; 465_{—}862del1398; 633delG; 1302del1302; 379_{—}433del55; 128_{—}129delC; 142_{—}143delA; 180delC; 209delA; 227_{—}228delC; 47_{—}651del605; 153_{—}536del384; exon 2-3del; exon 2-3del; exon 2-3del; exon 2-4-del; 562_{—}563delG; exon 4del; exon 4del; exon 4del; exon 4del; 610_{—}623del14; 837delC; 464_{—}465del2GGinsTTTGCT; 75_{—}76del2&insT; 125_{—}127insGG; 584_{—}585insT; 704_{—}705insA; 152_{—}153insCT; 842_{—}843insC; 649_{—}650insG; 127_{—}128insGG; 979_{—}980insAG; 165_{—}166insT; exon 6del; 1039_{—}1040insG; 735-2A>T; 5982AT; 465-1G>A; 465-1G>T; 291-2A>T; 921-1G>A; 597+1G>T; 143_{—}144>T; 841_{—}842>T; and 271_{—}272GG>TT.

9. The method of any one of claims 1 to 8 wherein the deletion, insertion or mutation of LKB1 is in the catalytic kinase domain.

10. The method of any one of claims 1 to 9 wherein the deletion, insertion or mutation of LKB1 is in codons 50-337.

11. The method of any one of claims 1 to 10 wherein the deletion, insertion or mutation of LKB1 causes a truncated LKB1 protein.

12. The method of any one of claims 1 to 11, wherein said mammal is human.

13. The method of any one of claims 1 to 12, wherein said cancer is a solid tumor cancer.

14. The method of any of claims 1 to 13 wherein said cancer is non-small cell lung carcinoma (NSCLC).

15. The method of any one of claims 1 to 14, wherein said cancer is pancreatic cancer.

16. The method of any one of claims 1 to 15 wherein the MEK inhibitor comprises a compound of Structure (I): 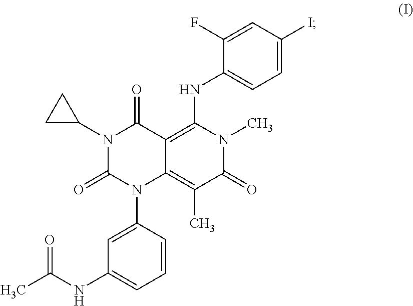 [SMILES: CC(=O)Nc1cccc(-n2c(=O)n(C3CC3)c(=O)c3c(Nc4ccc(I)cc4F)n(C)c(=O)c(C)c32)c1]
or a pharmaceutically acceptable salt or solvate thereof.

17. The method of any one of claims 1 to 16 wherein the mTOR inhibitor is selected from rapamycin, rapalogs, everolimus, deforolimus, and temsirolimus

18. The method of any one of claims 1 to 17 wherein said tumor cell also has at least one Braf mutation.

19. The method of claim 18 wherein said Braf mutation is selected from: R462I, I463S, G464V, G464E, G466A, G466E, G466V, G469A, G469E, D594V, F595L, G596R, L597V, L597R, T5991, V600E, V600D, V600K, V600R, T119S, and K601E.

20. The method of any one of claims 1 to 19 wherein said mammal shows a complete response to a therapeutically effective amount at least one MEK inhibitor and at least one mTOR inhibitor.

21. A method of treating a mammal in need thereof with a therapeutically effective amount of at least one MEK inhibitor and at least one mTOR inhibitor comprising:
determining if said mammal has at least one mutation in at least one Ras protein or gene encoding at least one Ras protein and at least one mutation, deletion and/or insertion in LKB1; and
administering a therapeutically effective amount of at least one MEK inhibitor and at least one mTOR inhibitor to said mammal if said mammal has at least one mutation in at least one Ras protein or gene encoding at least one Ras protein and does not have at least one mutation, deletion and/or insertion in LKB1.

22. A method for treating a human subject for cancer comprising the steps of:
obtaining at least one first sample from said subject;
detecting at least one mutation in at least one Ras protein or gene encoding at least one Ras protein in said at least one first sample from said subject;
optionally obtaining at least one second sample from said subject;
detecting at least one LKB1 mutation, deletion and/or insertion from at least one said first sample or at least one said optional second sample from said subject, and
treating said subject with a therapeutically effective amount of at least one MEK inhibitor and at least one mTOR inhibitor if at least one Ras mutation and at least one LKB1 mutation, deletion and/or insertion is detected in said first and/or second sample.

23. The method of claim 22 wherein said first sample and said second sample are the same sample.

24. The method of claim 22 or 23 wherein said first sample is a tumor sample.

25. The method of claim 22 or 23 wherein said first sample is a blood sample.

26. The method of claim 23 wherein said first sample and said second sample are different samples.

27. The method of claim 26 wherein said first sample is a tumor sample and said second sample is a blood sample.

28. The method of any one of claims 22 to 27 further comprising the step of correlating the subject's increased likelihood of response to treatment with at least one MEK inhibitor and at least one mTOR inhibitor if said subject has at least one mutation in at least one Ras protein or gene encoding at least one Ras protein or at least one mutation, deletion or insertion in LKB1.

29. The method of any one of claims 22 to 28, wherein said cancer is a solid tumor cancer.

30. The method of any of claims 22 to 29 wherein said cancer is non-small cell lung carcinoma (NSCLC).

31. The method of any one of claims 22 to 29, wherein said cancer is pancreatic cancer.

32. The method of any one of claims 22 to 31 wherein the MEK inhibitor comprises a compound of Structure (I): 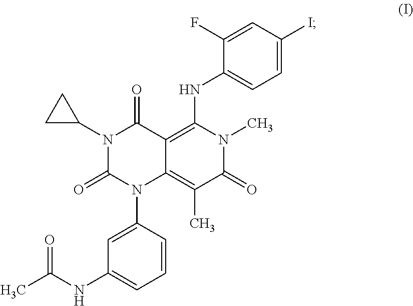 [SMILES: CC(=O)Nc1cccc(-n2c(=O)n(C3CC3)c(=O)c3c(Nc4ccc(I)cc4F)n(C)c(=O)c(C)c32)c1]
or a pharmaceutically acceptable salt or solvate thereof.

33. The method of any one of claims 22 to 32 wherein the mTOR inhibitor is everolimus.

34. A method of treating a human with cancer comprising
detecting at least one mutation in a Ras protein or a gene encoding at least one Ras protein from a sample from said human;
detecting the presence or absence of at least one mutation, deletion or insertion in LKB1 from a sample from said human; and
treating said human with a pharmaceutical composition comprising at least one MEK inhibitor comprising a compound of Structure (I): 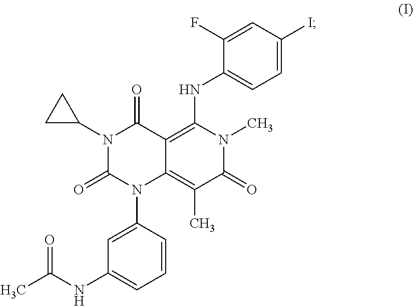 [SMILES: CC(=O)Nc1cccc(-n2c(=O)n(C3CC3)c(=O)c3c(Nc4ccc(I)cc4F)n(C)c(=O)c(C)c32)c1]
or a pharmaceutically acceptable salt or solvate thereof if at least one mutation in at least Ras protein or a gene encoding at least one Ras protein is detected and at least one mutation, deletion or insertion in LKB1 from a sample from said human is not detected.

35. The method of claim 34 further comprising detecting the present or absence of at least one BRG1 mutation.

36. The method of claim 34 or 35 further comprising treating said human with a therapeutically effective amount of at least one mTOR inhibitor.

37. The method of any one of claims 33 to 36 wherein Structure (I) is in the sodium salt form.

38. The method of any one of claims 33 to 36 wherein Structure (I) is in the form of a dimethyl sulfoxide solvate.

39. A method of treating a human with non-small cell lung carcinoma (NSCLC) wherein said human has at least one mutation in at least one Ras protein or gene encoding at least one Ras protein comprising administering to said human a therapeutically effective amount of a pharmaceutical composition comprising at least one MEK inhibitor comprising a compound of Structure (I): 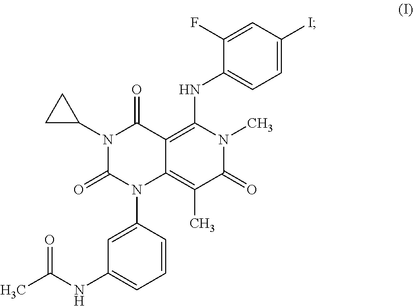 [SMILES: CC(=O)Nc1cccc(-n2c(=O)n(C3CC3)c(=O)c3c(Nc4ccc(I)cc4F)n(C)c(=O)c(C)c32)c1]
or a pharmaceutically acceptable salt or solvate thereof and at least one mTOR inhibitor.

40. The method of claim 39 wherein the Ras mutation is selected from: G125, G12V, G12D, G12A, G12C, G12R, G12F, G13C, G13A, G13D, V141, G60E, Q61K, Q61H, Q61R, T74P, E76G, E76K, E76Q, and A146T.

41. The method of claim 39 or 40 wherein said mTOR inhibitor is everolimus.

42. A method of treating a human with NSCLC wherein said human has at least one mutation in at least one Ras protein or gene encoding at least one Ras protein comprising administering to said human a therapeutically effective amount of a pharmaceutical composition comprising at least one MEK inhibitor comprising a compound of Structure (I): 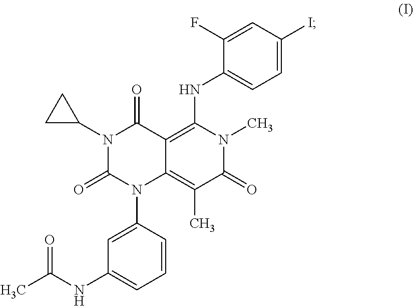 [SMILES: CC(=O)Nc1cccc(-n2c(=O)n(C3CC3)c(=O)c3c(Nc4ccc(I)cc4F)n(C)c(=O)c(C)c32)c1]
or a pharmaceutically acceptable salt or solvate thereof and at least one additional agent selected from the group of: docetaxel, PI3k/mTOR inhibitor, dasatinib, AKT inhibitor and everolimus.

43. The method of claim 42 wherein said PI3k/mTOR inhibitor is 2,4-difluoro-N-{2-(methyloxy)-5-[4-(4-pyridazinyl)-6-quinolinyl]-3-pyridinyl}benzenesulfonamide or a pharmaceutically acceptable salt or solvate thereof.

44. The method of claim 42 wherein said AKT inhibitor is N-{(1S)-2-amino-1-[(3-fluorophenyl)methyl]ethyl}-5-chloro-4-(4-chloro-1-methyl-1H-pyrazol-5-yl)-2-thiophenecarboxamide or a pharmaceutically acceptable salt or solvate thereof.
